(19) United States
(12) Patent Application Publication
(10) Pub. No.: US 2025/0235330 A1
(43) Pub. Date: Jul. 24, 2025

(54) PROSTHETIC ASSISTIVE HAND DEVICE WITH SPLIT HOOK AND OPPOSABLE THUMB

(71) Applicant: Touch Bionics Limited, Livingston (GB)

(72) Inventors: Benjamin Bokser, Holliston, MA (US); Rodrigo Mercader Rivera, Livingston (GB); Carlos Martinez-Luna, Holliston, MA (US); Sangwoo Park, Holliston, MA (US); Kevin Keough, Holliston, MA (US); Kevin Lawrence, Holliston, MA (US); Todd Farrell, Holliston, MA (US); Juliette Lou Marie Marchalot, Livingston (GB)

(21) Appl. No.: 19/022,663

(22) Filed: Jan. 15, 2025

Related U.S. Application Data
(60) Provisional application No. 63/623,036, filed on Jan. 19, 2024.

(51) Int. Cl. A61F 2/58; A61F 2/50; A61F 2/68

(52) U.S. Cl. CPC A61F 2/588; A61F 2002/5038; A61F 2002/5072; A61F 2002/587; A61F 2002/6836; A61F 2002/6854

(57) ABSTRACT

A prosthetic assistive device configured to attach to a distal end of a natural or prosthetic arm or wrist. The device may include a palm portion. A split hook may be coupled to a distal end of the palm portion. The split hook may comprise a first end effector and a second end effector. The prosthetic device may include a third end effector rotatably coupled to the palm portion at a proximal end of the palm portion. The third end effector may rotate about a transverse axis to pinch and unpinch, as well as rotate about a longitudinal axis to adduct and abduct. A rotating member such as a ring may rotate about a plate for adduction/abduction. The prosthetic device may include a drive assembly configured to rotate one or both of the split hook end effectors about two different axes.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

For example, this application claims priority to U.S. Provisional Patent Application No. 63/623,036, titled “PROSTHETIC ASSISTIVE HAND DEVICE WITH SPLIT HOOK AND OPPOSABLE THUMB” and filed Jan. 19, 2024, the entirety of which is incorporated herein by reference for all purposes and forms a part of this specification.

### GOVERNMENT SUPPORT

This invention was made with government support under 5R44HD090800-03 awarded by The National Institutes of Health: Eunice Kennedy Shriver National Institute of Child Health & Human Development. The government has certain rights in the invention. 37 CFR 401.14(f)(4).

### BACKGROUND

### Field

This disclosure relates to prosthetic assistive devices, in particular to a prosthetic assistive hand device with a drive assembly, split hook, and opposable thumb, which can emulate the pinching ability of a hand using the opposable thumb in multiple rotational positions, and a first rotation of the split hook as well as the grasping and pulling ability of a natural hand with a second rotation of the split hook, among other features.

### Description of the Related Art

Prosthetic assistive devices may be used to replace a corresponding amputated natural body part, such as a natural hand. A prosthetic assistive hand device can be designed for partially restoring the functionality of the natural hand and/or the functionality of a natural hand manipulating a simple tool, such as pliers or hooks. The use of prosthetic assistive devices such as typical split hook type devices have limited functionality and ease of use.

Able body individuals having use of two natural hands may switch between using their hands directly and using tools while performing a variety of tasks (e.g. the use of steel hook tools and pliers allows someone to perform tasks that would be too dangerous or too aggressive for natural human fingers). The use of tools by an amputee wearing a multi-articulating prosthetic hand presents challenges. Some prosthetic hands may be replaced with a prosthetic assistive hand device. Existing solutions for such devices have limited functionality as mentioned and also are complex such that removing the prosthetic hand and installing the prosthetic device, and vice versa, are not simple to do.

Improvements to these and other drawbacks of existing solutions for prosthetic assistive devices are therefore desirable.

### SUMMARY

The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods for prosthetic upper limb devices.

The following disclosure describes non-limiting examples of some embodiments. Other embodiments of the disclosed systems and methods may or may not include the features described herein. Moreover, disclosed advantages and benefits can apply only to certain embodiments of the invention and should not be used to limit the disclosure.

In one aspect, a prosthetic assistive device comprises a palm portion extending along a longitudinal axis and configured to attach to a distal end of a natural or prosthetic arm or wrist, a split hook comprising a first end effector and a second end effector, wherein the first end effector and the second end effector are configured to rotate about a first axis to cause a pinching or unpinching rotation, and wherein the first and/or second end effectors are configured to rotate about a second axis perpendicular to the first axis to open or close relative to each other, a drive assembly configured to cause the split hook to rotate about the first and second axes, and a thumb assembly coupled to a proximal end of the palm portion, the thumb assembly comprising a third end effector, wherein the third end effector is configured to rotate about a transverse axis to move between a retracted position and an extended position, and wherein the third end effector is configured to rotate about the longitudinal axis to adduct and abduct.

The various aspects may have various embodiments. For example, in the above and other aspects, the third end effector is configured to rotate to a plurality of discrete positions between the retracted position and the extended position; the third end effector is configured to rotate about the longitudinal axis to a plurality of discrete positions between an abducted position and an adducted position; the thumb assembly further comprises a rotation assembly coupled to the proximal end of the palm portion, wherein the third end effector is coupled to the rotation assembly, and wherein the rotation assembly is configured to rotate about the longitudinal axis to rotate the third end effector about the longitudinal axis, the rotation assembly comprises a locking mechanism configured to prevent or inhibit rotation of the rotation assembly when the locking mechanism is in a locked position, and/or the rotation assembly further comprises a plate coupled with the proximal end of the palm portion, and an annular rotating member rotatably coupled with the plate and the palm portion; the third end effector is rotatably coupled to the annular rotating member; the rotating member comprises a ratchet member, and the third end effector is rotatably coupled to the ratchet member; the ratchet member is configured to maintain the end effector in a plurality of discrete positions between the retracted position and the extended position; the third end effector comprises a cover slidably positioned over a body; the third end effector comprises a biasing member configured to bias the cover of the third end effector towards the ratchet member; the drive assembly comprises a pair of motors, a pair of motor gears coupled to the motors, a pair of worm gear assemblies, a pair of driven gear assemblies, and a bevel gear, wherein the pair of worm gear assemblies are positioned so an axis of rotation of the pair of worm gear assemblies extends from a dorsal side of the prosthetic device to a palm side of the prosthetic device, the pair of motors are configured to rotate the pair of driven gear assemblies in a same first direction to cause a pinch rotation of the split hook, and to rotate the pair of driven gear assemblies in a same second direction opposite the first direction to cause an unpinch rotation of the split hook, and/or the pair of motors are configured to rotate the pair of driven gear assemblies in opposite directions relative to each other to open and/or close the first and second end effectors relative to each other; the device further comprises a release mechanism configured to disengage the drive assembly; the release mechanism comprises a lever and a cam, wherein the lever is configured to rotate the cam between an engaged position and a disengaged position; in the engaged position a recess in the cam faces away from a worm gear of the drive assembly and the cam prevents or inhibits translation of a worm gear of the drive assembly, and wherein in the disengaged position, the recess in the cam faces the worm gear of the drive assembly so the worm gear translates into the recess of the cam; the locking mechanism comprises a cam configured to rotate between the locked position and an unlocked position; the rotation assembly comprises a detent mechanism positioned in the plate, wherein the detent mechanism is configured to extend into a recess of a plurality of recesses in the annular rotating member in order to maintain the third end effector in one of a plurality of discrete positions between an abducted position and an adducted position; and/or the detent mechanism comprises a ball and spring.

In another aspect, a prosthetic assistive device comprises a palm portion, a split hook comprising a first end effector and a second end effector, a third end effector and a drive assembly. The palm portion is configured to attach to a distal end of a natural or prosthetic arm or wrist. The split hook is coupled to a distal end of the palm portion. The third end effector is rotatably coupled to the palm portion at a proximal end of the palm portion. The drive assembly is configured to rotate the split hook about two axes.

In another aspect, a prosthetic assistive device comprises a palm portion, a split hook comprising a first end effector and a second end effector, a third end effector, a drive assembly. The palm portion is configured to attach to a distal end of a natural or prosthetic arm or wrist. The split hook is coupled to a distal end of the palm portion. The first and/or second end effectors are configured to rotate about a first axis to cause a pinching or unpinching rotation and to rotate about a second axis perpendicular to the first axis to open or close relative to each other. The third end effector is rotatably coupled to the palm portion and is configured to rotate about a third axis parallel to the first axis to rotate toward and away from the split hook. The drive assembly is configured to cause the split hook to rotate about the first and second axes.

The various aspects may have various embodiments. For example, in the above and other aspects, in some embodiments the third end effector is configured to stow into a recess formed in a palm side of the palm portion and/or the third end effector comprises a plurality of rotatable segments.

In another aspect, a prosthetic assistive device is configured to attach to a distal end of a natural or prosthetic arm or wrist. The prosthetic assistive device comprises a split hook and a drive assembly. The split hook comprises a first end effector and a second end effector. The drive assembly comprises a pair of motors, a pair of motor gears coupled to the motors, a pair of worm gear assemblies, a pair of driven gear assemblies, and a bevel gear. The pair of worm gear assemblies are positioned so an axis of rotation of the pair of worm gear assemblies extends from a dorsal side of the prosthetic device to a palm side of the prosthetic device. The drive assembly is configured to rotate the split hook about two axes.

The various aspects may have various embodiments. For example, in the above and other aspects, in some embodiments: the motors are configured to rotate the pair of driven gear assemblies in a same first direction to cause a pinch rotation of the split hook, and to rotate the pair of driven gear assemblies in a same second direction opposite the first direction to cause an unpinch rotation of the split hook; the motors are configured to rotate the pair of driven gear assemblies in opposite directions relative to each other to open and/or close the first and second end effectors relative to each other; the first end effector is configured to rotate to open and/or close the split hook; the first end effector and the second end effector are configured to rotate to open and/or close the split hook; the prosthetic device further comprises a third end effector rotatably coupled to a palm portion of the prosthetic assistive device; the third end effector is configured to rotate toward and away from the split hook; and/or further comprising a proximal connector configured to attach to the prosthetic wrist or arm.

In another aspect, an upper limb prosthetic comprises the prosthetic assistive device. In some embodiments, the upper limb prosthetic comprises a prosthetic arm or prosthetic wrist.

In another aspect, a method of operating a prosthetic assistive device is described. The method comprises rotating first and second end effectors about a first axis to cause a pinching or unpinching rotation of the first and second end effectors; rotating the first end effector relative to the second end effector about a second axis that is angled with respect to the first axis to increase or decrease an angle between the first and second end effectors; and rotating a third end effector about a third axis toward or away from the first and second end effectors.

In some embodiments, the method of operating the prosthetic assistive device further comprises rotating the first end effector relative to the second end effector about the second axis to form a gap between the first and second end effectors, and rotating the third end effector relative to the first and second end effectors with the third end effector located in the gap between the first and second end effectors; rotating a first motor and a second motor in the same direction and at the same speed to cause the first and second end effectors to rotate about the first axis only; rotating a first motor and a second motor in the same direction but at different speeds to cause the first and second end effectors to rotate about the first and second axes simultaneously; rotating a first motor and a second motor in opposite directions at the same speed to cause the first and second end effectors to rotate about the second axis only; and/or rotating first and second driving bevel gears to cause rotation of a driven bevel gear to cause rotation of the first and/or second end effector about the first and/or second axis.

As further example, in the above and other aspects, in some embodiments: a prosthetic assistive device may include three end effectors; the prosthetic assistive device may provide multiple points to improve stability and security of the grip of the prosthetic assistive device; the prosthetic assistive device may combine the benefits of a tool with the emulation of a hand grip (i.e., an opposable thumb); the use of objects (i.e., handles or instruments) designed to be grasped by a hand may be grasped against a palm of the prosthetic assistive device to improve stability when compared to grabbing with a pinching grip of a split hook device; the end effectors of the device may be swapped so a user may select an end effector to use from a variety of interchangeable end effectors; the prosthetic assistive device may reduce or minimize the degree of body positioning and adjustment required by the user to complete a task; the prosthetic assistive device may reduce or minimize body positioning and adjustment from the most comfortable position in order to decrease the likelihood of repetitive strain injuries, (e.g., a user may develop shoulder problems if the elbow needs to be elevated above the shoulder to complete a frequent task); and/or the prosthetic assistive device may vary an angle of a plane of operation for the split hook grasp, to allow the user to comfortably approach a position for a given task.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated and make part of this disclosure.

FIG. 1A illustrates a perspective view of an example prosthetic assistive device, having a split hook with two end effectors, and an “opposable thumb” embodied as a retractable third end effector.

FIG. 1B is a dorsal view of the prosthetic assistive device of FIG. 1A.

FIG. 1C is a bottom or palm view of the prosthetic assistive device of FIG. 1A.

FIG. 1D is a side view of the prosthetic assistive device of FIG. 1A, with a third end effector in an extended position.

FIG. 1E shows the prosthetic assistive device of FIG. 1D, with the third end effector in a retracted position.

FIGS. 2A-2F illustrate opening and closing motions of the split hook end effectors of the prosthetic assistive device of FIG. 1A.

FIGS. 3A to 3E illustrate pinching and unpinching motions of the split hook end effectors of the prosthetic assistive device of FIG. 1A.

FIG. 4A is a perspective palm view of the prosthetic assistive device of FIG. 1A with extended third end effector.

FIGS. 4B to 4D are detail views of the third end effector of FIG. 4A.

FIGS. 5A-5E illustrate rotational motions of the third end effector of the prosthetic assistive device of FIG. 1A, rotating between an extended position and a retracted/stowed position.

FIGS. 6A-6C illustrate the prosthetic assistive device of FIG. 1A with the split hook and third end effector rotating between an open, unpinched configuration and a closed, pinched configuration.

FIGS. 7A-7F illustrate the prosthetic assistive device of FIG. 1A with the split hook and third end effector rotating to grasp object of various sizes, with the third end effector rotating between an opening between the first and second end effectors of the split hook.

FIGS. 8A-8C illustrate the prosthetic assistive device of FIG. 1A in various grasping configurations.

FIGS. 9A-9D illustrate are various views of a drive assembly that may be used with the prosthetic assistive device of FIG. 1A.

FIGS. 10A-10E illustrate the drive assembly rotating a split hook of the prosthetic assistive device of FIG. 9A around a first lateral axis in a first direction via rotation of both motors in the same first motor rotation direction.

FIGS. 11A-11E illustrate the drive assembly rotating the split hook of the prosthetic assistive device of FIG. 9A around the first lateral axis in a second, opposite direction, via rotation of both motors in the same second motor rotation direction that is opposite the first motor rotation direction.

FIGS. 12A-12D illustrate the drive assembly rotating one of the end effectors of the split hook of the prosthetic assistive device of FIG. 9A outwardly around a second axis in a first direction to open the split hook, via rotation of the motors in opposite directions or rotation of the motors in a same first motor direction but at different speeds.

FIGS. 13A-13D illustrate the drive assembly rotating one of the end effectors of the split hook of the prosthetic assistive device of FIG. 9A inwardly around the second vertical axis in a second direction to close the split hook, via rotation of the motors in opposite directions or rotation of the motors in a same second motor direction opposite the first motor direction but at different speeds.

FIG. 14A illustrates the prosthetic assistive device of FIG. 1A and a prosthetic wrist.

FIG. 14B illustrates the prosthetic assistive device of FIG. 1B and a prosthetic arm.

FIG. 15 illustrates a perspective view of another example prosthetic assistive device, having an “opposable thumb” embodied as a rotatable third end effector.

FIG. 16A illustrates a perspective view of a thumb assembly of the prosthetic assistive device of FIG. 15 in isolation.

FIG. 16B illustrates the thumb assembly of FIG. 16A with a cover of the thumb assembly removed.

FIG. 16C illustrates a rotating member of a rotation assembly of the thumb assembly of FIG. 16A in isolation.

FIG. 16D illustrates a plate of the rotation assembly of the thumb assembly of FIG. 16A in isolation.

FIG. 16E illustrates a cross-section of the plate of the rotation assembly of the thumb assembly of FIG. 16A in isolation.

FIG. 16F. illustrates the rotation assembly of FIG. 16A with the plate shown in cross-section.

FIGS. 17A-17G illustrate top views of the prosthetic assistive device of FIG. 15 showing rotation of the opposable thumb about a longitudinal axis.

FIGS. 18A and 18B illustrate the prosthetic assistive device of FIG. 15 with the opposable thumb in a middle and retracted position.

FIG. 18C illustrates the prosthetic assistive device of FIG. 15 with the opposable thumb in an extended position.

FIGS. 19A-19D illustrate sequential views of the prosthetic assistive device of FIG. 15 showing rotating of the opposable thumb about a transverse axis.

FIG. 20A illustrates the prosthetic assistive device of FIG. 15 with a distal end of the opposable thumb in contact with a distal end of a first end effector of a split hook of the prosthetic assistive device.

FIG. 20B illustrates the prosthetic assistive device of FIG. 15 with the distal end of the opposable thumb in contact with a distal end of a second end effector of the split hook of the prosthetic assistive device.

FIGS. 21A and 21B illustrate side views of the prosthetic assistive device of FIG. 15 in a gripping position.

FIG. 21C illustrates the prosthetic assistive device of FIG. 15 in the gripping position shown in FIGS. 21A and 21B, with the prosthetic assistive device holding a tool.

FIGS. 22A and 22B illustrate the prosthetic assistive device of FIG. 15 with an object grasped by and positioned between the opposable thumb and the split hook.

FIGS. 22C and 22D illustrate the prosthetic assistive device of FIG. 15 with the distal end of the opposable thumb in between the distal ends of the end effectors of the split hook.

FIG. 23 illustrates another example thumb assembly for a prosthetic assistive device in isolation.

FIG. 24A illustrates a top perspective view of another example plate for a rotation assembly of a prosthetic assistive device shown in isolation with a locking cam in an unlocked position.

FIG. 24B illustrates the locking cam of the plate of FIG. 24A in isolation.

FIG. 24C illustrates a bottom perspective view of the plate of FIG. 24A in the unlocked position.

FIG. 24D illustrates a top perspective view of the plate of FIG. 24A in a locked position.

FIG. 24E illustrated a bottom perspective view of the plate of FIG. 24A in the locked position.

FIG. 25A illustrates a cross-section of the prosthetic device of FIG. 15 with a release mechanism in an engaged position.

FIG. 25B illustrates a cross-section of the prosthetic device of FIG. 15 with the release mechanism in a disengaged position.

FIG. 25C-25E illustrate partial cross-sectional views of the prosthetic device of FIG. 15 with the release mechanism in the engaged position.

FIG. 25F illustrates a partial cross-sectional view of the prosthetic device of FIG. 15 with the release mechanism in the engaged position.

FIGS. 25G and 25H illustrate a cam of the release mechanism of the prosthetic device of FIG. 15.

FIG. 26A illustrates a perspective view of another example prosthetic assistive device, having a channel extending through a palm wall of the prosthetic assistive device.

FIG. 26B illustrates a front view of the prosthetic device of FIG. 26A.

FIG. 26C illustrates a bottom-side perspective view of the prosthetic device of FIG. 26C.

### DETAILED DESCRIPTION

The following detailed description is directed to certain specific embodiments of the development. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments.

Systems, devices and methods for prosthetic assistive hand devices are described herein that are simple and robust, with less chance of damage and less visual obstruction when compared to typical solutions. A user may perform a variety of tasks, e.g., manipulating food items while cooking, or holding work pieces while performing arts and crafts or trade jobs, etc.

In some embodiments, a prosthetic assistive device may include a first end effector and a second end effector. The end effectors may move with two degrees of freedom. A first degree of freedom may include flexion-extension (e.g. pinching and unpinching) and a second degree of freedom may include abduction-adduction (e.g. opening and closing) of one or both of the end-effectors. In some embodiments, a tilting head in a drive assembly may generate the flexion-extension movement (i.e., a hand like movement). The flexion-extension movement may move the end effector(s) towards or away from a palm of the prosthetic assistive device.

In some embodiments, the flexion-extension movement may be generated by two worm wheels rotating in a same direction. The two worm wheels may be locked in rotation to their respective rotations by driving bevel gears. In some embodiments, if the two worm wheels rotate at the same speed (i.e., zero rotational speed relative to each other), forces transmitted by each of the two worm wheels to a driven bevel gear coupled to the tilting head may be the same and may be transmitted to the driven bevel gear in the same direction relative to a first axis of rotation of the driven bevel gear and the tilting head. Accordingly, the forces may not generate a force couple and the driven gear may not rotate about the second axis of rotation (e.g. a “palm-dorsal” axis or a “vertical” axis as used herein). The driven gear may transmit the forces from the two worm wheels to the tilting head to rotate the tilting head about a first axis of rotation (e.g. a “lateral” axis as used herein).

In some embodiments, the abduction-adduction movement may be generated by the worm wheels rotating in opposite directions relative to each other. The magnitude of the rotation of each gear relative to each other may be twice the magnitude of the rotational speed relative to the housing. Forces applied to the driven bevel gear by the two driven gear assemblies may oppose each other relative to the axis of rotation of the driven bevel gear (e.g., the second axis of rotation). Accordingly, the forces applied to the driven bevel gear may not be aligned. Therefore, the forces may create a force couple around the second axis of rotation of the driven bevel gear causing the driven bevel gear to rotate around the second axis of rotation.

In some embodiments, the two end effectors may be removably coupled to the tilting head. Accordingly, the two end effectors may be removed and replaced with new end effectors. In some embodiments, one or more motors may rotate the worm gears. In some embodiments, a mechanical locking mechanism may be added to the driven bevel gear to prevent or inhibit rotation of the driven bevel gear in the second axis of rotation.

In some embodiments, the driven bevel gear and the tilting head may be rotated around both the first axis and the second axis at the same time. In some embodiments, rotation about both the first and the second axis may be generated when the magnitude of the rotational speed of the worm gears relative to each other do not match and the zero or twice opposite speed conditions are not met. For example, the worm gears may both rotate in the same first direction but at different speeds to cause an opening and pinching motion, and may both rotate in the same opposite, second direction but at different speeds to cause a closing and unpinching motion. In some embodiments, a user may select between flexion-extension exclusively, abduction-adduction exclusively and/or combined rotation or motion.

In some embodiments, a motor/planetary gearbox, a bevel pair, a worm gear, and a worm wheel may form a transmission chain. The transmission chain may generate rotation of the bevel driving gears that apply forces to the driven bevel gear. In other embodiments, any of the components of the transmission chain may include spur or helical gears. In some embodiments, the transmission chain may be supported by bearings (i.e., ball bearings or plain bearings). In some embodiments, a cross member may be positioned between the driving gears. The cross member may enhance rigidity of the transmission chain. The cross member may be coupled to both the driving gears so the cross member does not prevent or inhibit rotation of the driving gears.

The above and other features will now be described in further detail with reference to the drawings.

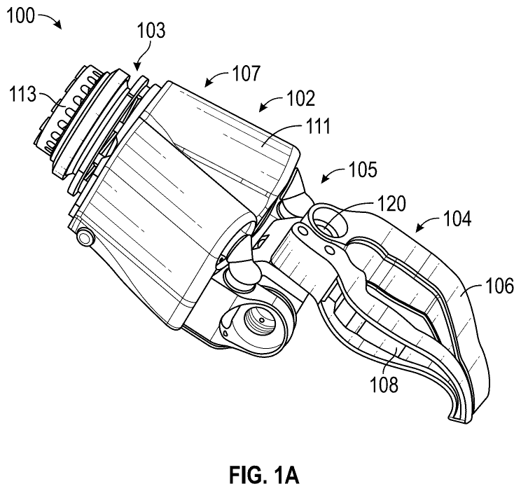

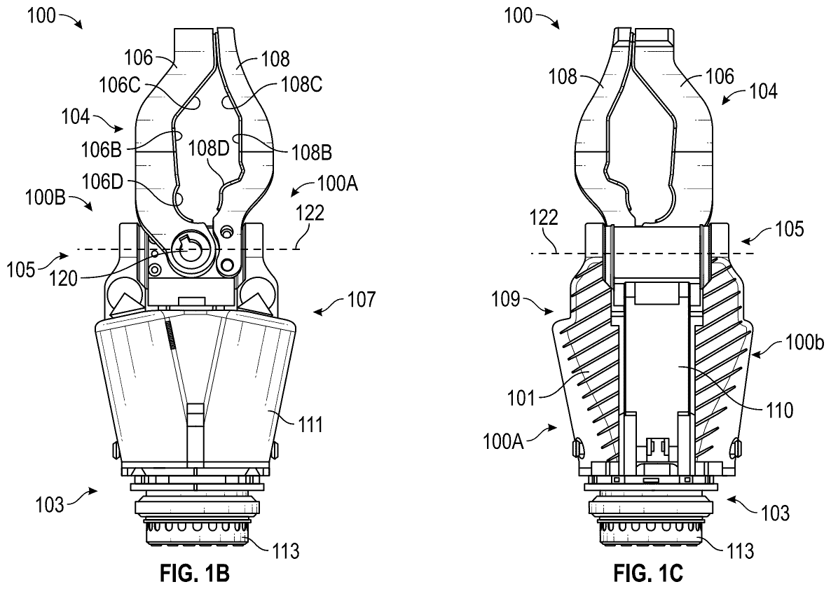

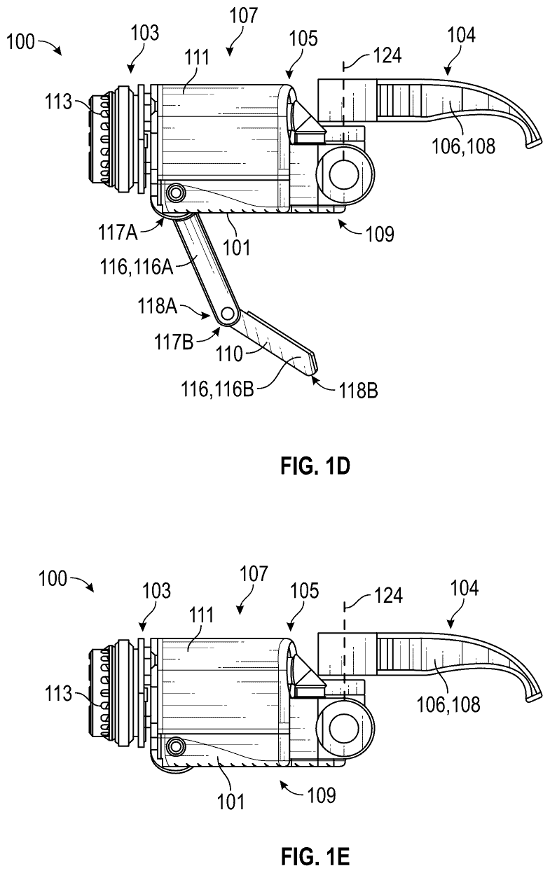

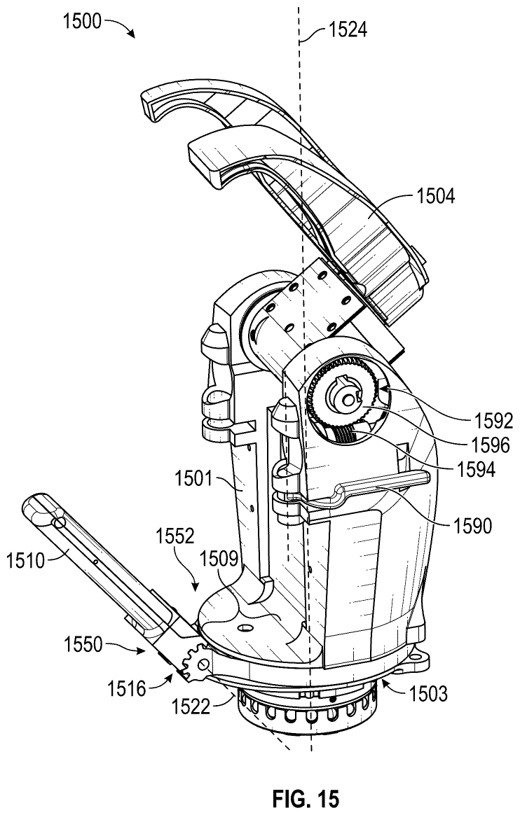

FIGS. 1A-1E illustrate various views of an example prosthetic assistive hand device or “prosthetic device” 100 having a split hook with two end effectors, and an “opposable thumb” embodied as a retractable third end effector. In particular, FIG. 1A illustrates a perspective view, FIG. 1B is a top or dorsal view, FIG. 1C is a bottom or palm view, FIG. 1D is a side view with the third end effector in an extended position, and FIG. 1E shows the prosthetic device with the third end effector in a retracted position. Any features shown in or described with respect to FIGS. 1A-14B can be included with any of the systems, devices or methods as shown in or described with respect to FIGS. 15-26C, and vice versa.

The prosthetic device 100 may include a palm portion 102. The palm portion 102 may extend from a proximal end 103 to a distal end 105. The palm portion 102 may include a dorsal side 107 and a palm side 109 opposite the dorsal side 107. In some embodiments, the palm portion 102 may include a housing 111 and a palm wall 101. One or more internal components (e.g., the motors 902 and the drive assembly 910) of the prosthetic device 100 may be positioned in the housing 111. The housing 111 may be configured to provide structure and rigidity. Accordingly, the housing 111 may form an exoskeleton of the prosthetic device 100. The housing 111 may allow the prosthetic device 100 to not include a chassis. In some embodiments, the housing 111 may prevent or inhibit damage to the one or more internal components of the prosthetic device 100. In some embodiments, the housing 111 may prevent or inhibit impact of the one or more internal components of the prosthetic device 100. In some embodiments, the housing 111 may prevent or inhibit dust, dirt, water, and/or debris from contacting the one or more internal components. In some embodiments, the housing 111 may include a metal alloy and/or any other suitable rigid material. In some embodiments, the housing 111 may formed by 3D printing, casting, and/or any other machining process.

The prosthetic device 100 may include a split hook 104. The split hook 104 may be coupled to the palm portion 102 at the distal end 105 of the palm portion 102. The split hook 104 may be coupled to a tilting head 120 positioned at a distal end 105 of the palm portion 102. In some embodiments, the split hook 104 may be removably coupled to the tilting head 120. Accordingly, the split hook 104 may be decoupled from the tilting head 120 and replaced with a second split hook 104. A user may remove the split hook 104 via a multi-tool, a screwdriver, a small pry bar and/or any other tool. The split hook 104 may be from about 3 inches to about 8 inches or from 3 to 8 inches in overall length, or from about 4 inches to about 6 inches or from 4 to 6 inches. The split hook 104 may be from about 1 inch to about 4 inches or from 1 to 4 inches, or from about 1.5 inches to about 3 inches or from 1.5 to 3 inches in overall width in the closed configuration.

In some embodiments, the split hook 104 may include a first end effector 106 and/or a second end effector 108. The end effectors may be elongated, structural members of the split hook 104 configured to interact with a surrounding environment, such as grasping, touching, or pointing at objects. The end effectors 106, 108 may be coupled to the tilting head 120 as described above. One or both end effectors 106, 108 may rotate about a first vertical or palm-dorsal axis 122 to pinch and unpinch (also called flexion and extension herein). One or both end effectors 106, 108 may rotate about a second lateral axis 124 to open and close (also called abduction and adduction herein). The end effectors 106, 108 may perform pairs of rotations simultaneously, e.g. pinch and open, pinch and close, unpinch and open, or unpinch and close.

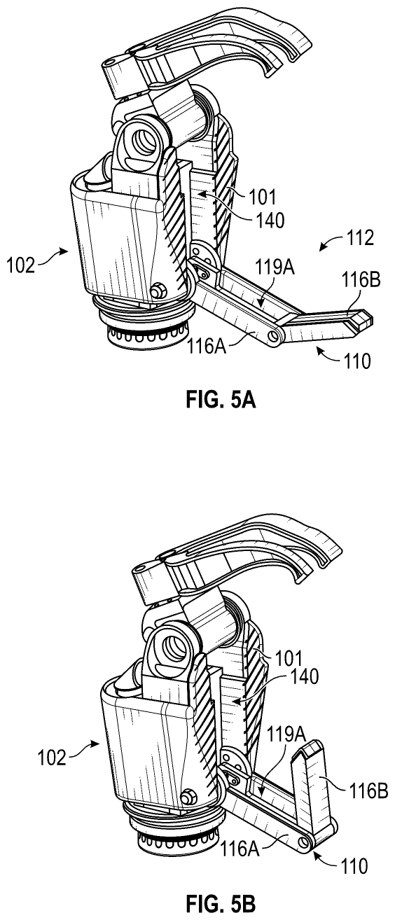

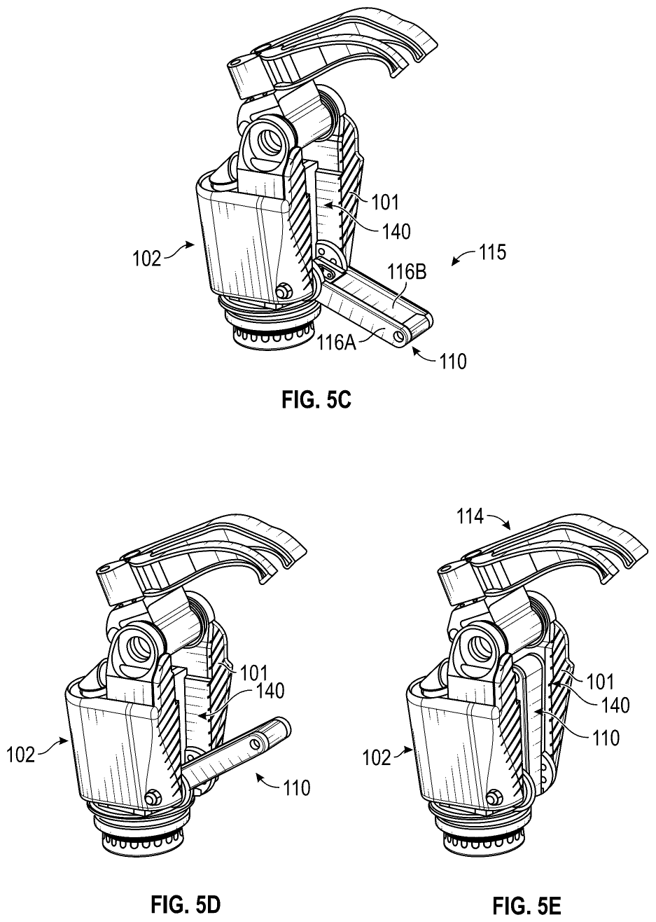

In some embodiments, the prosthetic device 100 may include a third end effector 110. The third end effector 110 may form an “opposable thumb” of the prosthetic device 100. The third end effector 110 may be coupled to the palm portion 102 on the palm side 109 of the palm portion 102. The third end effector 110 may be coupled to the palm portion 102 at the proximal end 103 of the palm portion 102. In some embodiments, the third end effector 110 may be coupled to the palm portion 102 a distance from (e.g., near) the proximal end 103 of the palm portion 102. In some embodiments, the third end effector 110 may be coupled to the palm portion 102 at the distal end 105 of the palm portion 102. In some embodiments, the third end effector 110 may be coupled to the housing 111. In some embodiments, the third end effector 110 may be coupled to the housing 111 by a plate coupled to the housing 111 at a proximal end of the palm portion 102. “Coupled” as used in this disclosure includes both direct and indirect connections between the coupled parts. The third end effector 110 may be rotated to an extended position 112, as shown in FIG. 1D, and to a retracted position 114, as shown in FIGS. 1C and 1E, as described further below with reference to FIGS. 5A-5E. The third end effector 110 may be from about 1 inch to about 6 inches or from 1 to 6 inches, or from about 2 inches to about 4 inches or from 2 to 4 inches, in overall linear length (e.g., in a straight configuration for embodiments with rotatable segments).

In some embodiments, the third end effector 110 may include one or more segments 116, which may be rotatably coupled together. The third end effector 110 may include one (1) segment 116, two (2) segments 116, and/or three (3), or more, segments 116. In some embodiments, the third end effector 110 may include a first segment 116A and a second segment 116B. The first segment 116A may include a first proximal end 117A and a second distal end 118A opposite the first end 117A. The second segment 116B may include a first proximal end 117B and a second distal end 118B. The first proximal end 117A of the first segment 116A may be rotatably coupled to the palm portion 102. The first proximal end 117B of the second segment 116B may be rotatably coupled to the second distal end 118A of the first segment 116A.

In some embodiments, the prosthetic device 100 may be coupled to and/or integrated with a user's natural or prosthetic arm and/or wrist, e.g. a socket thereof. The prosthetic device 100 may be coupled with a prosthesis coupled to a user's arm. The prosthesis may be coupled to the user by condyle suspension, suction, and/or harnessing. Accordingly, the prosthesis may secure the prosthetic device 100 to an arm missing a hand and/or a portion of the arm. In some embodiments, the prosthetic device 100 may be coupled to a prosthetic arm. In some embodiments, the prosthetic device 100 may include a connector 113. The connector 113 may extend from the proximal end 103 of the palm portion 102. The connector 113 may provide for rotation of the prosthetic device 100 in relation to the user's arm. The connector 113 may include a wrist disarticulation attachment, wrist flexion attachment, powered wrist rotation attachment, and/or any other wrist connector or attachment device. The connector 113 may attach to a prosthetic wrist or arm. The connector 113 may be a prosthetic wrist. In some embodiments, the type of the connector 113 may be based on a length of a missing portion of the user's arm, preferences of the user, and/or requirements of the user.

In some embodiments, the split hook 104 may move with two degrees of freedom. The split hook 104 may rotate around the first axis 122 shown in FIGS. 1B and 1C, and a second axis 124 shown in FIGS. 1D and 1E. The first axis 122 may extend in a lateral direction across the prosthetic device 100 from a medial side 100A of the prosthetic device 100 to an opposite lateral side 100B of the prosthetic device 100. The second axis 124 may extend in a direction through split hook 104 and/or the tilting head 120 towards the first axis 122. The second axis 124 may be a “vertical” axis for sake of description only, as the second axis 124 need not remain vertical, for example when the tilting head 120 rotates. The second axis 124 may be angled with respect to the first axis 122. The second axis 124 may be perpendicular to the first axis 122. The second axis 124 may intersect the first axis 122. The second axis 124 may not intersect, for example be offset from, the first axis 122. Accordingly, when the split hook 104 is in an extended position, the second axis 124 may extend in a direction from the dorsal side 107 to the palm side 109 of the palm portion 102. As described further below, when the split hook 104 rotates about the first axis 122, the split hook 104 may rotate between the extended (e.g., unpinched) position and a pinched position. The second axis 124 may rotate about the first axis 122 with the split hook 104 when the split hook 104 rotates between the extended position and the pinched position.

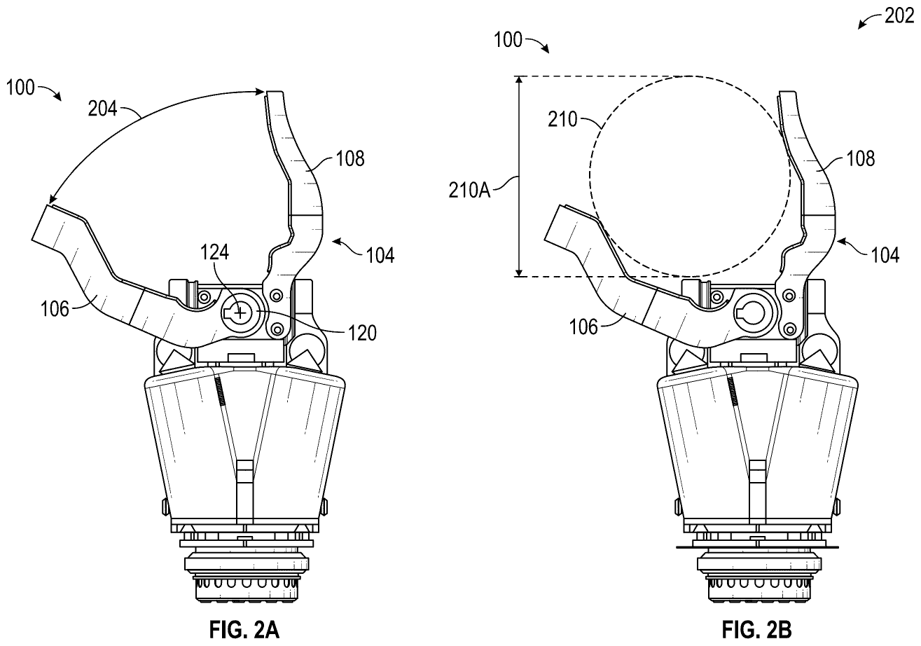

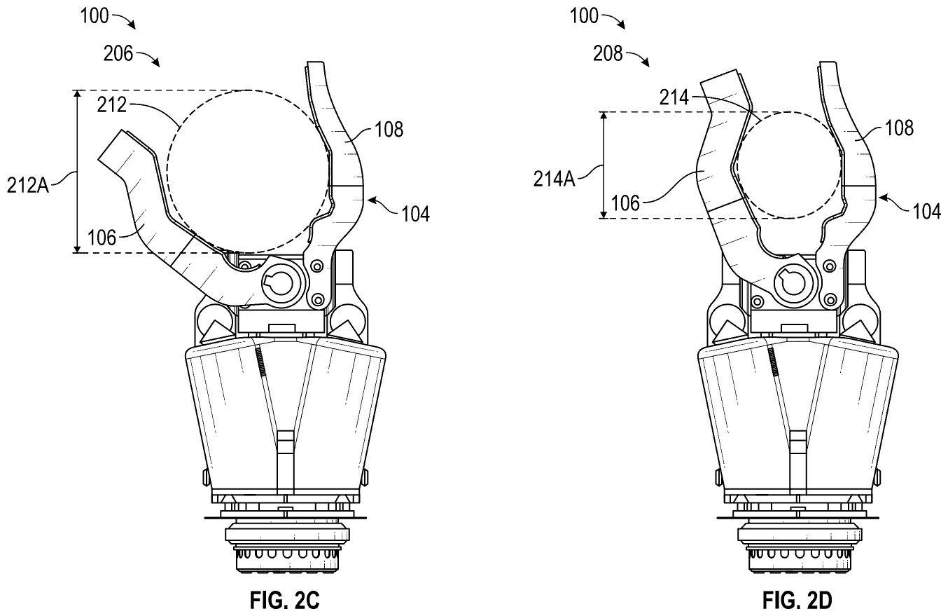

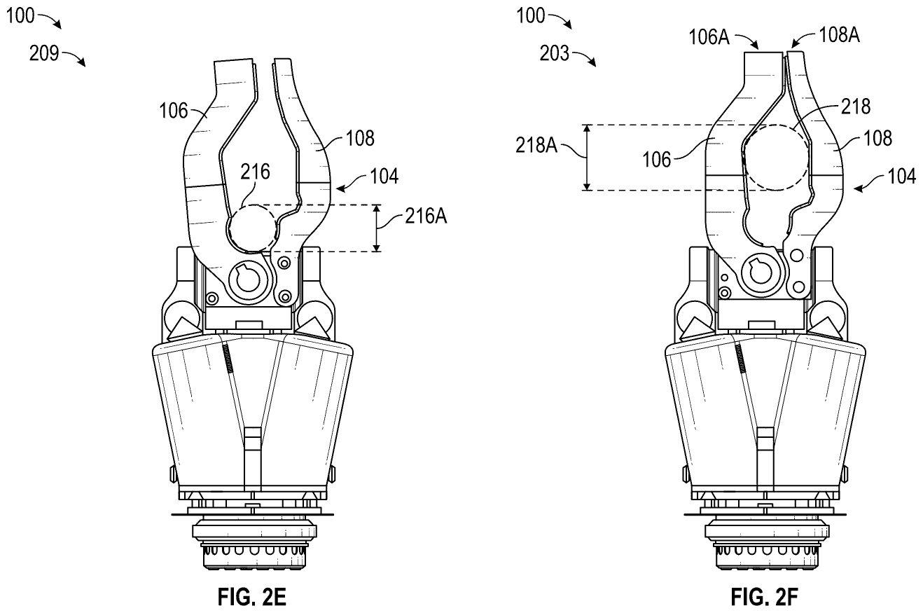

FIGS. 2A-2F illustrate sequential dorsal views of the split hook 104 performing opening (abduction) and closing (adduction) motions of the end effectors 106, 108. The end effectors 106, 108 are shown in various open configurations in FIGS. 2A-2E, and in a closed configuration 203 in FIG. 2F.

As shown in FIG. 2A, when the split hook 104 is in the open position 202, the first end effector 106 may form an angle 204 with the second end effector 108. The angle may be from 130 to 30 degrees, or from 110 to 45 degrees, or from 90 to 45 degrees, or at least 30 degrees, or at least 45 degrees, or at least 60 degrees. In some embodiments, the first end effector 106 may rotate about the second axis 124 when the split hook 104 rotates between the open position 202 and the closed position 203. In some embodiments, the tilting head 120 may rotate about the second axis 124 in order to rotate the split hook 104 between the open position 202 and the closed position 203. As shown, in some embodiments, the first end effector 106 may rotate about the second axis 124 in a first direction (counterclockwise, as oriented in the figure) and the second end effector 108 may remain stationary (for example relative to a fixed reference frame, such as the palm portion). Thus only one end effector 106 may move.

In some embodiments, the first end effector 106 may rotate about the second axis 124 in a first direction (e.g., counterclockwise, as oriented in the figure) and the second end effector 108 may rotate in a second direction about the second axis 124 opposite the first direction (e.g., clockwise, as oriented in the figure). In some embodiments, the second end effector 108 may rotate about the second axis 124 in a first direction (e.g., clockwise, as oriented in the figure) and the first end effector 106 may remain stationary.

As shown in FIGS. 2B-2F, the split hook 104 may be configured to rotate to one or more intermediate positions between the open position 202 and the closed position 203. It is to be appreciated that although the split hook 104 is described as rotating to one or more intermediate positions between the open position 202 and the closed position 203, the split hook 104 may rotate to any position between the open position 202 and the closed position 203. The angle 204 between the first end effector 106 and the second end effector 108 may increase or decrease to move between the open position 202 and the closed position 203. As shown in FIG. 2B, when the split hook 104 is in the open position 202 (which may be similar to the position shown in FIG. 2A) the split hook 104 may be configured to hold a first object 210 with a first size 210A, which may be a width or diameter of the object. For example, the first size 210A may include a diameter from about 65 to 75 millimeters, or about 72 millimeters.

As shown in FIG. 2C, the split hook 104 may rotate to a first intermediate position 206 between the open position 202 and the closed position 203. When the split hook 104 is in the first intermediate position 206, the angle 204 between the first end effector 106 and the second end effector 108 may be smaller than the angle 204 when the split hook 104 is in the open position 202. When the split hook 104 is in the first intermediate position 206, the split hook 104 may be configured to hold a second object 212 with a second size 212A smaller than the first size 210A of the first object 210. For example, the second size 212A may include a diameter from about 50 to 65 millimeters, or about 60 millimeters.

As shown in FIG. 2D, the split hook 104 may rotate to a second intermediate position 208 between the open position 202 and the closed position 203. When the split hook 104 is in the second intermediate position 208 the angle 204 between the first end effector 106 and the second end effector 108 may be smaller than the angle 204 when the split hook 104 is in the first intermediate position 206. When the split hook 104 is in the second intermediate position 208 the split hook 104 may be configured to hold a third object 214 with a third size 214A smaller than the second size 212A of the second object 212. For example, the third size 214A may include a diameter from about 30 to 45 millimeters, or about 40 millimeters.

As shown in FIG. 2E, the split hook 104 may rotate to a third intermediate position 209 between the open position 202 and the closed position 203. When the split hook 104 is in the third intermediate position 209 the angle 204 between the first end effector 106 and the second end effector 108 may be smaller than the angle 204 when the split hook 104 is in the second intermediate position 208. When the split hook 104 is in the third intermediate position 209 the split hook 104 may be configured to hold a fourth object 216 with a fourth size 216A smaller than the third size 214A of the third object 214. For example, the fourth size 216A may include a diameter from about 12 to 22 millimeters, or about 18 millimeters.

As shown in FIG. 2F, in some embodiments, when the split hook 104 is in the closed position 203, the distal end 106A of the first end effector 106 and the distal end 108A of the second end effector 108 may contact each other so the angle 204 between the first end effector 106 and the second end effector 108 includes an angle of zero (0) degrees. In some embodiments, the first end effector 106 and/or the second end effector 108 may be sized and/or shaped so the first end effector 106 and the second end effector 108 form a gap therebetween when the split hook 104 is in the closed position 203. The split hook 104 may be configured to hold a fifth object 218 in the gap when the split hook 104 is in the closed position 203 having a fifth size 218A. The fifth size 218A may include a diameter may be from about 22 to 30 millimeters, or about 25 millimeters.

As shown in FIGS. 2B-2F, the objects 210, 212, 214, 216, 218 may contact any portion of the first end effector 106 and/or the second end effector 108 when the split hook 104 holds the objects 210, 212, 214, 216, 218. In some embodiments, the point of contact between the objects 210, 212, 214, 216, 218 and the first end effector 106 and/or the second end effector 108 may be based on the angle 204 between the first end effector 106 and the second end effector 108.

Additionally, the contours of the inner, opposing surfaces of the end effectors 106, 108 provide for grasping the various sized objects in the various positions. As shown in FIG. 1B for example, the inner contour of the end effector 106 may include a proximal portion 106D having a rounded, outwardly recessed contour, which may be radiused. The proximal portion 106D may have a contour that extends into the end effector 106. A central portion 106B extends distally from the proximal portion 106D and may have a substantially linear contour, that may be extending perpendicular to the first axis 122 in the closed position as shown. A distal portion 106C extends distally from the central portion 106B to a tip portion and may have a linear contour that is angled inwardly with respect to the central portion 160B as shown. The end effector 108 may have an inner contour with a proximal portion 108D that juts into the gap between the end effector 106, 108, such as extending distally and then laterally outwardly. A central portion 108B extends distally from the proximal portion 108D and may be a substantially linear contour that may be extending perpendicular to the first axis 122 in the closed position as shown, which may be parallel to the central portion 106B in such position. A distal portion 108C extends distally from the central portion 108B to a tip portion and may have a contour that is substantially linear and angled inwardly with respect to the central portion 108B. The tip portions of the end effectors 106, 108 may be distal ends configured to contact each other in the closed position as shown.

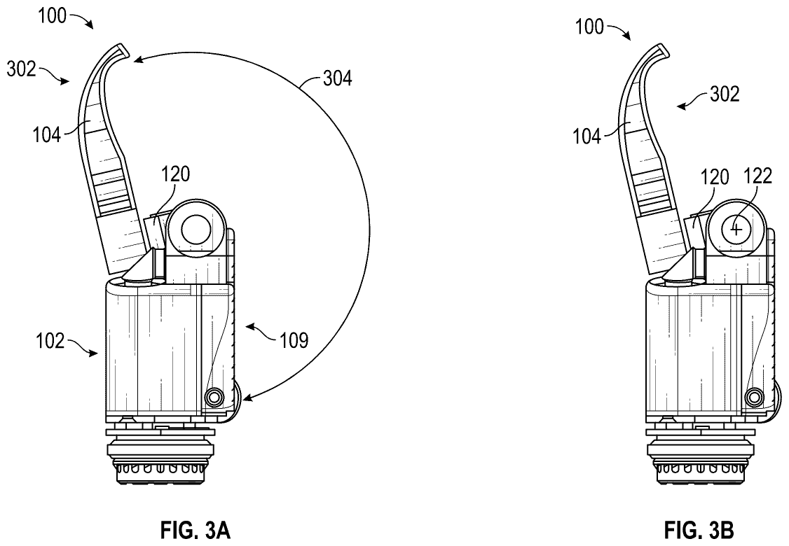

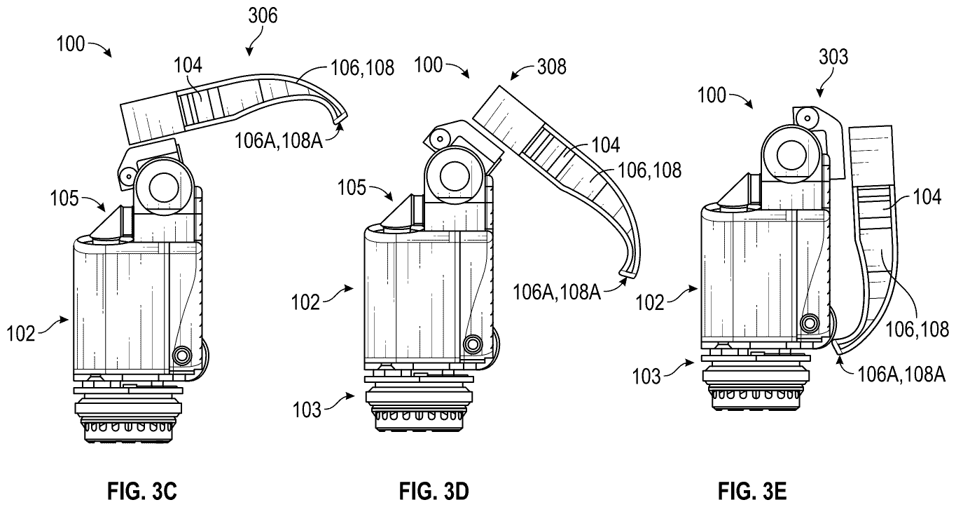

FIGS. 3A-3E illustrate pinching and unpinching motions of the split hook end effectors of the prosthetic device 100. A medial view of the prosthetic device 100 is shown with the split hook 104 rotating between an extended position 302, as shown in FIGS. 3A and 3B, and a pinched position 303, as shown in FIG. 3E. The split hook 104 may rotate about the first axis 122 when the split hook 104 rotates between the extended position 302 and the pinched position 303. In some embodiments, the tilting head 120 may rotate about the first axis 122 in order to rotate the split hook 104 between the extended position 302 and the pinched position 303. The split hook 104 may rotate about the first axis 122 in a direction towards the palm side 109 of the palm portion 102 to rotate to the pinched position 303. The split hook 104 may rotate about the first axis 122 in a direction away from the palm side 109 of the palm portion 102 to rotate to the extended position 302. The total angle 304 that the split hook 104 may rotate through may be from 90 to 220 degrees, from 100 to 200 degrees, from 110 to 190 degrees, at least 180 degrees, at least 150 degrees, or at least 120 degrees.

As shown in FIG. 3A, the split hook 104 may form an angle 304 with the palm side 109 of the palm portion 102 when the split hook 104 is in the extended position 302. In some embodiments, when the split hook 104 is in the extended position 302, the end effectors 106, 108 may extend distally from the palm portion 102. The end effectors 106, 108 may rotate about the first axis 122 together as a unit, as further described.

As shown in FIGS. 3C and 3D, the split hook 104 may be configured to rotate to one or more intermediate positions between the extended position 302 and the pinched position 303. Although the split hook 104 is described as rotating to one or more particular intermediate positions between the extended position 302 and the pinched position 303, the split hook 104 may rotate to any position between the extended position 302 and the pinched position 303.

As shown in FIG. 3C, the split hook 104 may rotate to a first intermediate position 306. When the split hook 104 in the first intermediate position 306, the angle 304 may be smaller than when the split hook 104 is in the extended position 302. In some embodiments, when the split hook 104 is in the first intermediate position 306, the distal ends 106A, 108A of the end effectors 106, 108 may be positioned distal to the distal end 105 of the palm portion 102.

As shown in FIG. 3D, the split hook 104 may rotate to a second intermediate position 308. When the split hook 104 is in the second intermediate position 308, the angle 304 may be smaller than when the split hook 104 is in the first intermediate position 306. In some embodiments, when the split hook 104 is in the second intermediate position 308, the distal ends 106A, 108A of the end effectors 106, 108 may be positioned between the distal end 105 of the palm portion 102 and the proximal end 103 of the palm portion 102.

As shown in FIG. 3E, when the split hook 104 is in the pinched position 303, the end effectors 106, 108 may extend towards the proximal end 103 of the palm portion 102 so the distal ends 106A, 108A of the end effectors 106, 108 are positioned at or near the proximal end 103 of the palm portion 102.

The split hook 104 may perform the rotations, or be in any of the positions, as described with respect to FIGS. 3A-3E while in a variety of opened or closed (abducted or adducted) configurations. For example, the split hook 104 may have any of the configurations shown in FIGS. 2A-2F while performing any of the rotations, or being in any of the positions shown, with respect to FIGS. 3A-3E.

In some embodiments, the end effectors 106, 108 may include a partial ellipse shape along a length of the end effectors 106, 108 when viewed from a side view, as shown in FIGS. 3A-3E. The partial ellipse shape of the end effectors 106, 108 may allow the end effectors 106, 108 to hold (i.e., grasp) a cylindrical object. Accordingly, a user may pull the cylindrical object. In some embodiments, the partial ellipse shape may allow the end effectors 106, 108 to hold (i.e., grasp) a cylindrical object with a diameter within a range of diameters In some embodiments, the end effectors 106, 108 may be sized and/or shaped to hold an object with a diameter of 1 mm to 30 mm, or about 20 mm. the end effectors 106, 108 may be sized and/or shaped to hold an object with a diameter up to 50 mm, or about 25 mm.

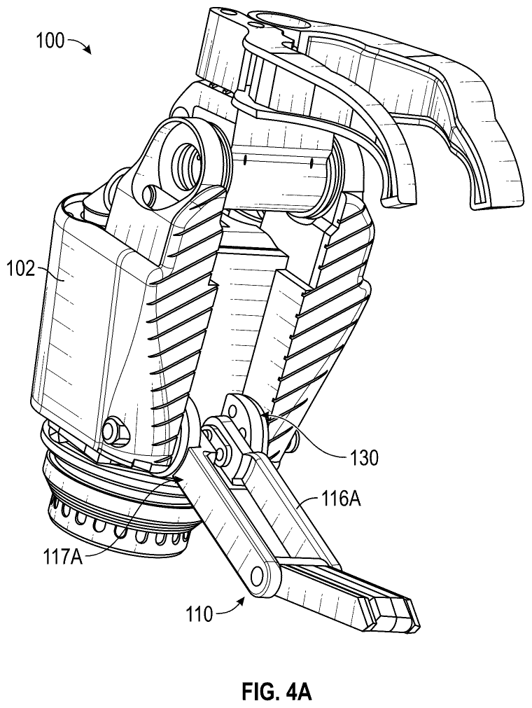

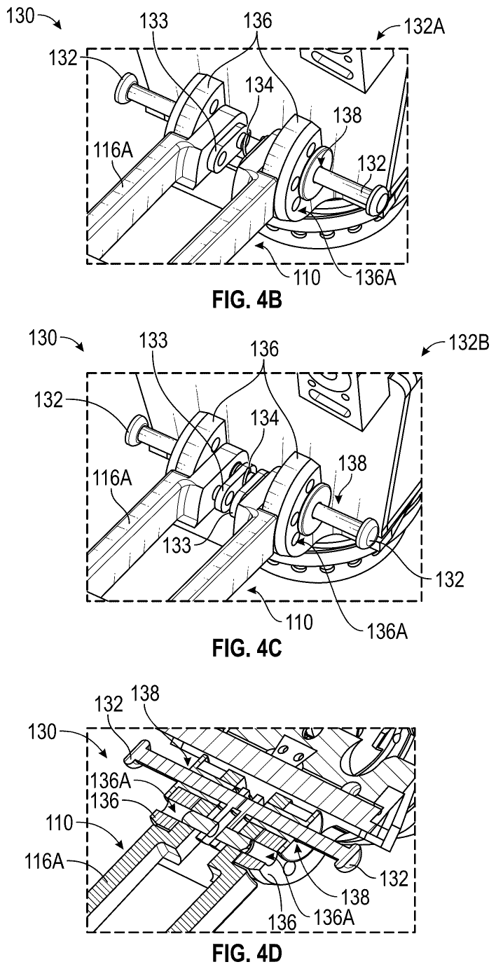

FIGS. 4A-4D illustrate details of the third end effector 110 of the prosthetic device 100. As shown in FIG. 4A, the first segment 116A of the third end effector 110 may be rotatably coupled to the palm portion 102. The first end 117A of the first segment 116A may be rotatably coupled to the palm portion 102 via a hinge assembly 130.

As shown in FIGS. 4B-4D, the hinge assembly 130 may include pins 132, a biasing member 134, and plates 136 (e.g., an index wheel). The pins 132 may extend through the plates 136 at pivot point 138. The pins 132 may be configured to axially rotate. The first segment 116A of the third end effector 110 may be coupled to the pins 132. Accordingly, when the pins 132 axially rotate, the first segment 116A may rotate about the pivot point 138.

In some embodiments, the pins 132 may include a locking portion 133. The locking portion 133 may be configured to extend through the first segment 116A and openings 136A in the plates 136. Accordingly, the locking portion 133 may prevent or inhibit rotation of the first segment 116A. In some embodiments, the pins 132 may be configured to axially translate through the plates 136 between a locked position 132A, as shown in FIG. 4B, and an unlocked position 132B. When the pins 132 are in the locked position 132A the locking portion 133 may extend through the first segment 116A and the openings 136A of the plates 136 to prevent or inhibit rotation of the first segment 116A. When the pins 132 are in the unlocked position 132B, the locking portion 133 may not extend into the openings 136A of the plates 136. Accordingly, when the pins 132 are in the unlocked position 132B, the first segment 116A may rotate about the pivot point 138. In some embodiments, a user may translate the pins 132 in a direction towards each other to move the pins 132 from the locked position 132A to the unlocked position 132B.

In some embodiments, the biasing member 134 may be positioned between the pins 132. The biasing member 134 may bias the pins 132 towards the locked position 132A. In some embodiments, the biasing member 134 may include a spring.

FIGS. 5A-5E illustrate the third end effector 110 rotating between the extended position 112, as shown in FIG. 5A, and the retracted position 114, as shown in FIG. 5E. In some embodiments, the palm portion 102 may include a recess 140 on the palm wall 101. The third end effector 110 may be positioned in the recess 140 when the third end effector 110 is in the retracted (e.g., stowed) position 114. The palm wall 101 may be heat and/or fireproof. Accordingly, the palm wall 101 may prevent or inhibit heat from internal components of the prosthetic device 100 from damaging the third end effector 110 and/or the hinge assembly 130.

As shown in FIG. 5A, when the third end effector 110 is in the extended position 112, the first segment 116A may extend in a direction away from the palm side 109 of the palm portion 102 and the second segment 116B may extend in a direction away from the palm side 109 of the palm portion 102. Accordingly, the third end effector 110 may not be positioned in the recess 140 when the third end effector 110 in the extended position 112.

As shown in FIGS. 5B and 5C, the third end effector 110 may be moved to an intermediate position 115. In some embodiments, when the third end effector 110 is in the intermediate position 115, the second segment 116B may be positioned in an opening 119A of the first segment 116A. The second segment 116B may be rotated in a direction towards the palm portion 102 to move the third end effector 110 to the intermediate position 115.

As shown in FIGS. 5D and 5E, the third end effector 110 may be moved from the intermediate position 115 to the retracted position 114. The first segment 116A may be rotated in a direction towards the recess 140 to move the third end effector 110 from the intermediate position 115 to the retracted position 114.

In some embodiments, the third end effector 110 may be moved manually by a user, for example by the user's other natural or prosthetic hand or device. In some embodiments, the prosthetic device 100 may include a motor configured to rotate the third end effector 110.

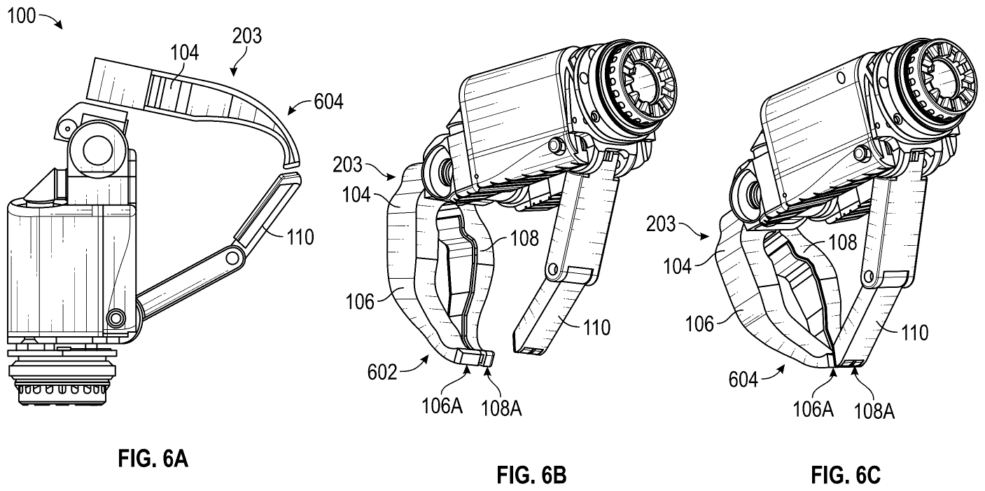

FIGS. 6A-6C illustrate the prosthetic device 100 with the split hook 104 in the closed position 203 and moving between a closed (adducted) and unpinched position 602, as shown in FIG. 6B, and a closed pinched position 604, as shown in FIGS. 6A and 6C. When the split hook 104 is in the closed unpinched position 602, the split hook 104 may be positioned so the distal ends 106A, 108A of the end effectors 106, 108 do not contact the third end effector 110. When the split hook 104 is in the closed pinched position 604, the split hook 104 may be positioned so the distal ends 106A, 108A of the end effectors 106, 108 contact the third end effector 110. In some embodiments, the split hook 104 may rotate about the first axis 122 (shown in FIGS. 1B and 1C) to move between the closed unpinched position 602 and the closed pinched position 604.

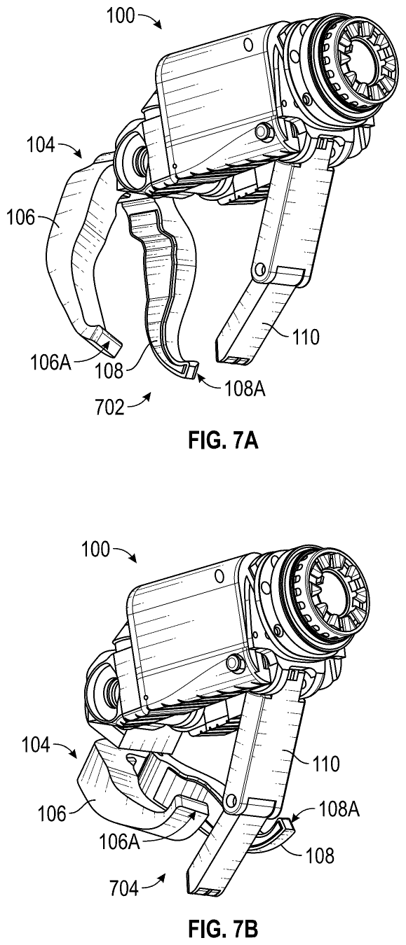

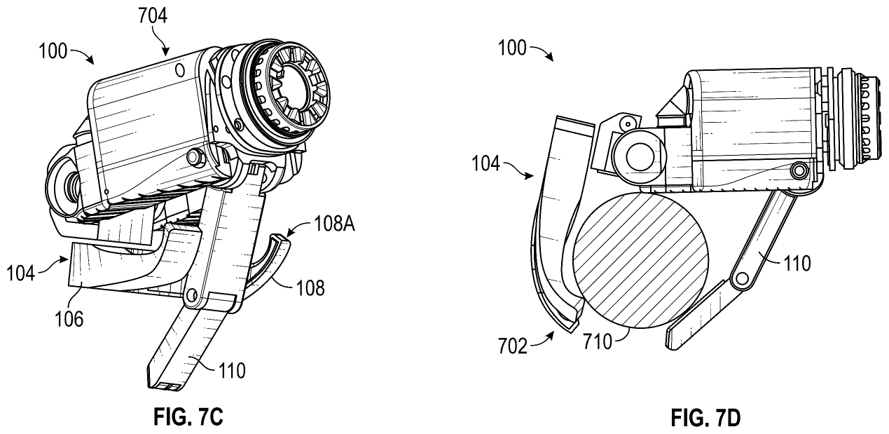

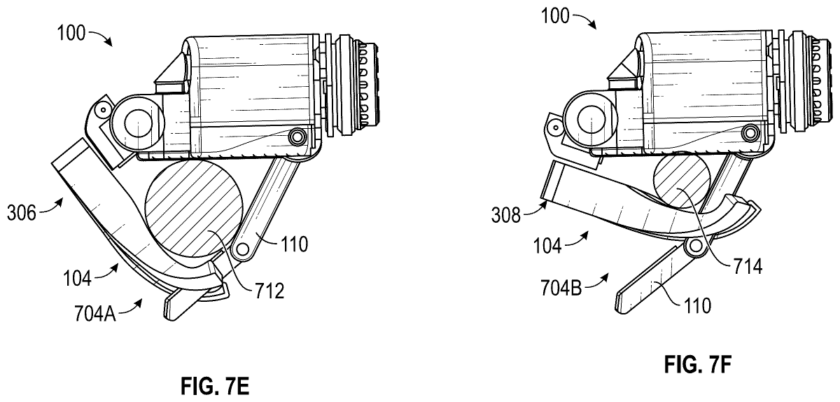

FIGS. 7A-7C illustrate sequential views of the prosthetic device 100 moving between an open (abducted) and unpinched position 702, as shown in FIG. 7A, and an open pinched position 704, as shown in FIGS. 7B and 7C. When the prosthetic device 100 is in the open unpinched position 702, the split hook 104 may be positioned so the distal ends 106A, 108A of the end effectors 106, 108 are positioned distally from the third end effector 110. When the prosthetic device 100 is in the open pinched position 704, the split hook 104 may be positioned so the end effectors 106, 108 extend past and/or overlap with the third end effector 110. In some embodiments, the split hook 104 may rotate about the first axis 122 (shown in FIGS. 1B and 1C) to move between the open unpinched position 702 and the closed unpinched position 704. In some embodiments, when the prosthetic device 100 is in the open unpinched position 702 and/or the open pinched position 704, the third end effector 110 may be positioned between the extended position 112 and the retracted position 114.

As shown in FIGS. 7D-7F, the prosthetic device 100 may be configured to hold an object between the split hook 104 and the third end effector 110 when the prosthetic device 100 is in the open unpinched position 702 and/or the open pinched position 704. As shown in FIG. 7D, when the prosthetic device 100 is in the open unpinched position 702, the prosthetic device 100 may be configured to hold a first object 710. As shown in FIG. 7E, when the prosthetic device 100 is in a first open pinched position 704A the prosthetic device 100 may be configured to hold a second object 712. As shown in FIG. 7F, when the prosthetic device 100 is in a second open pinched position 704B, the prosthetic device 100 may be configured to hold a third object 714. When the prosthetic device 100 is in the first open pinched position 704A, the split hook 104 may be positioned in the first intermediate position 306. When the split hook 104 is in the second open pinched position 704B, the split hook 104 may be positioned in the second intermediate position 308. In some embodiments, the first object 710 may be larger than the second object 712, and the second object 712 may be larger than the third object 714.

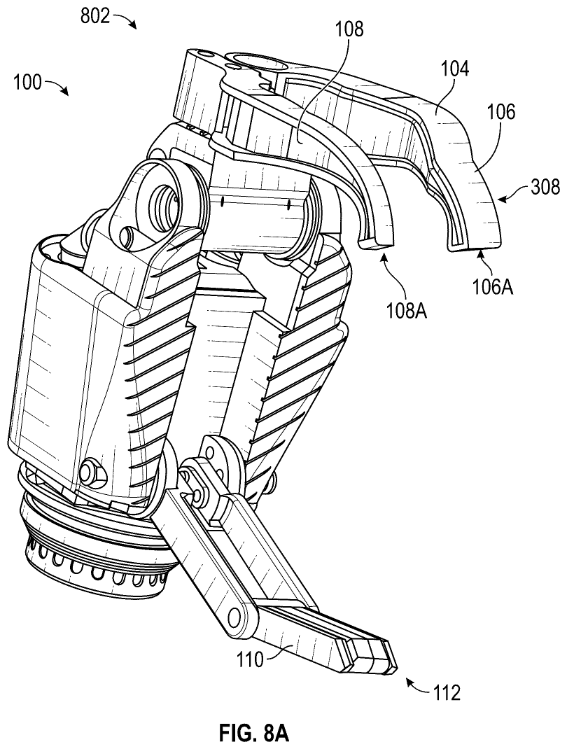

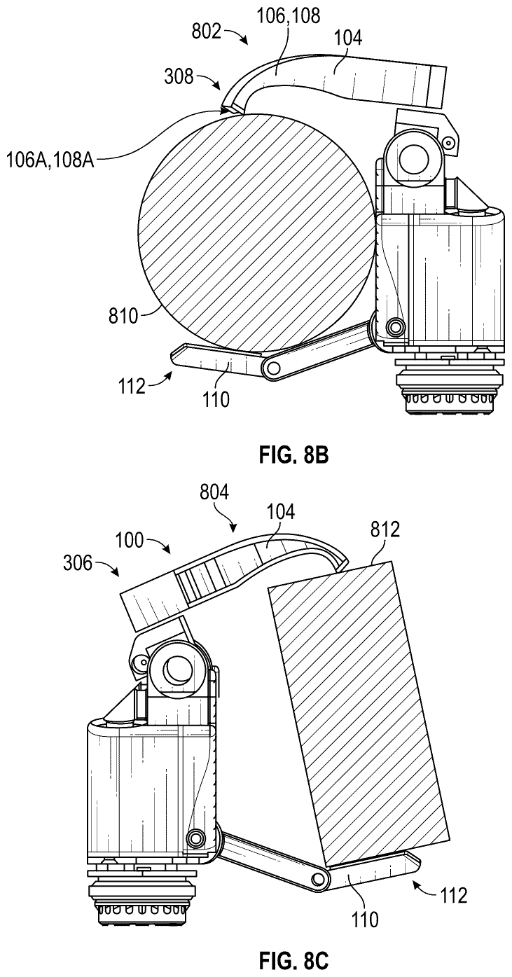

FIGS. 8A-8C illustrate the prosthetic device 100 moving between a first grasping position 802, as shown in FIGS. 8A and 8B, and a second grasping position 804, as shown in FIG. 8C. When the prosthetic device 100 is in the first grasping position 802 and/or the second grasping position 804, the third end effector 110 may be in the extended position 112. In some embodiments, when the prosthetic device 100 is in the first grasping position 802 the split hook 104 may be positioned in the second intermediate position 308. In some embodiments, when the prosthetic device 100 is in the second grasping position 804 the split hook 104 may be positioned in the first intermediate position 306. In some embodiments, the prosthetic device 100 is in the first grasping position 802 and/or the second grasping position 804, the prosthetic device 100 may be configured to hold an object between the split hook 104 and the third end effector 110 so the object contacts the distal ends 106A, 108A of the end effectors 106, 108. When the prosthetic device 100 is in the first grasping position 802 the prosthetic device 100 may be configured to hold a first object 810. When the prosthetic device 100 is in the second grasping position 804 the prosthetic device 100 may be configured to hold a second object 812. The first object 810 may be smaller than the second object 812. In any of the grasping positions, the split hook 104 may have the end effectors 106, 108 in an open position and not contacting each other as shown, or they may be closed and contacting each other.

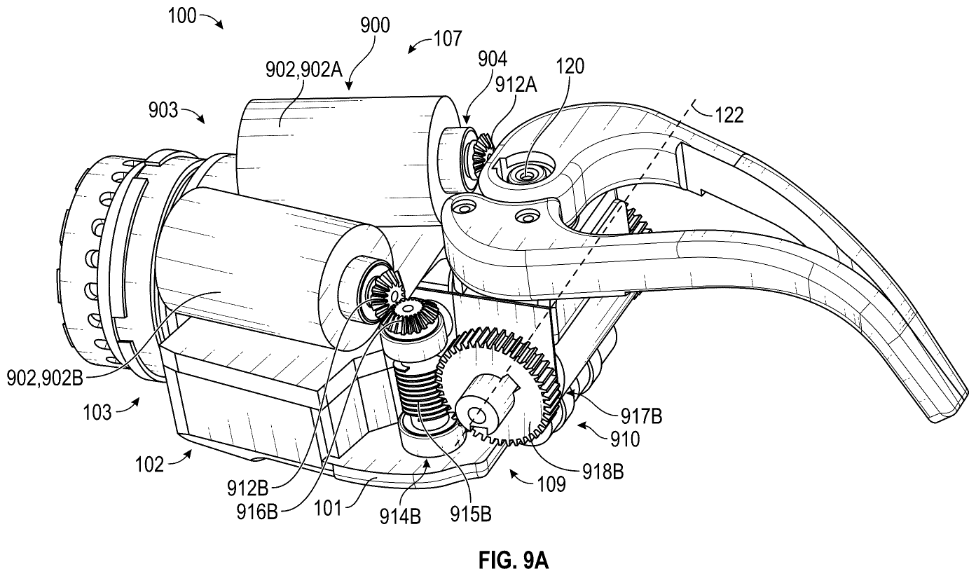

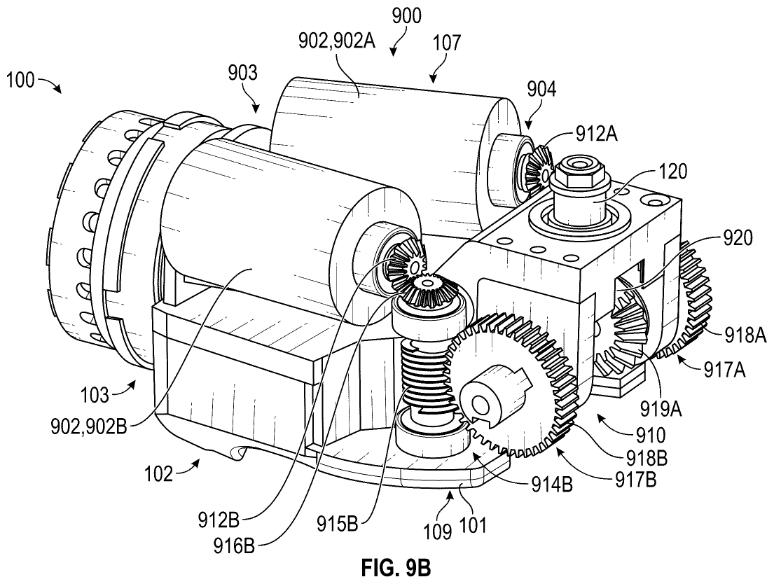

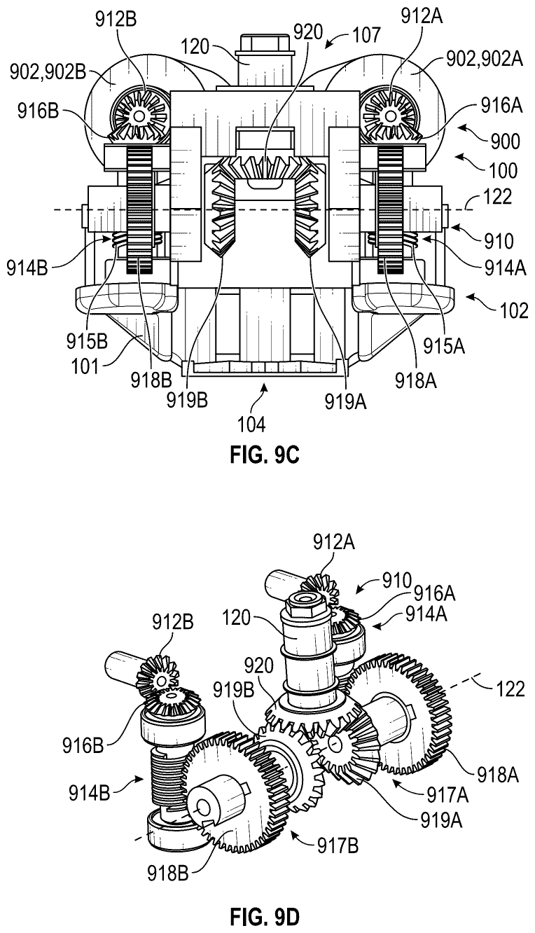

FIGS. 9A-9D illustrates internal components 900 of the prosthetic device 100. FIGS. 9A-C illustrate the prosthetic device 100 with the housing 111 removed from the palm wall 101. FIG. 9D illustrates a drive assembly 910 of the prosthetic device 100.

As shown in FIGS. 9A-9C, the prosthetic device 100 may include motors 902 and the drive assembly 910. In some embodiments, the motors 902 and the drive assembly 910 may be coupled and/or supported by the palm wall 101. In some embodiments, the prosthetic device 100 may include a first motor 902A and a second motor 902B. The motors 902 may be positioned on the dorsal side 107 of the palm portion 102. The motors 902 may be positioned so proximal ends 903 of the motors 902 are positioned closer to each other than distal ends 904 of the motors 902. Accordingly, the motors 902 may be positioned so the motors 902 extend away from each other. The position of the motors 902 may allow the prosthetic device 100 to be to be tapered in a direction towards the proximal end 103 of the palm portion 102. Accordingly, the prosthetic device 100 may resemble the shape of a human hand.

In some embodiments, the first motor 902A may include a first bevel gear 912A and the second motor 902B may include a second bevel gear 912B. The first bevel gear 912A may be coupled to the first motor 902A so the first bevel gear 912A rotates about an axis of rotation of the first motor 902A. The second bevel gear 912B may be coupled to the second motor 902B so the second bevel gear 912B rotates about an axis of rotation of the first motor 902A.

In some embodiments, the drive assembly 910 may include a first worm gear assembly 914A and a second worm gear assembly 914B. The first worm gear assembly 914A may be positioned so an axis of rotation of the first worm gear assembly 914A is perpendicular to the axis of rotation of the first motor 902A and the first bevel gear 912A. The second worm gear assembly 914B may be positioned so an axis of rotation of the second worm gear assembly 914B is perpendicular to the axis of rotation of the second motor 902B and the second bevel gear 912B. In some embodiments, the first worm gear assembly 914A and the second worm gear assembly 914B may be positioned so the first worm gear assembly 914A and the second worm gear assembly 914B extend in a direction from the dorsal side 107 to the palm side 109 of the palm portion 102.

The first worm gear assembly 914A may include a first worm gear 915A and a third bevel gear 916A. The third bevel gear 916A may be coupled to the first worm gear 915A such that when the third bevel gear 916A rotates, the first worm gear 915A axially rotates. The second worm gear assembly 914B may include a second worm gear 915B and a fourth bevel gear 916B. The fourth bevel gear 916B may be coupled to the second worm gear 915B such that when the third bevel gear 916A rotates, the 915A axially rotates. The third bevel gear 916A and the fourth bevel gear 916B may be positioned so the third bevel gear 916A and the fourth bevel gear 916B interact with the first bevel gear 912A and the second bevel gear 912B respectively. When the first bevel gear 912A rotates the first bevel gear 912A may rotate the third bevel gear 916A. When the second bevel gear 912B rotates the second bevel gear 912B may rotate the fourth bevel gear 916B.

In some embodiments, the drive assembly 910 may include a first driven gear assembly 917A and a second driven gear assembly 917B. The first driven gear assembly 917A and the second driven gear assembly 917B may be positioned so the first driven gear assembly 917A and the second driven gear assembly 917B rotate about the first axis 122. The first driven gear assembly 917A may include a first driven gear 918A and a first driven bevel gear 919A. The second driven gear assembly 917B may include a second driven gear 918B and a second driven bevel gear 919B. The first driven gear 918A and the second driven gear 918B may be configured to interact with the first worm gear 915A and the second worm gear 915B respectively. Accordingly, when the first worm gear 915A rotates, the first worm gear 915A may rotate the first driven gear 918A, and when the second worm gear 915B rotates, the second worm gear 915B may rotate the first driven gear 918A.

In some embodiments, the drive assembly 910 may include a tilting head bevel gear 920. The tilting head bevel gear 920 may be coupled to the tilting head 120. The tilting head bevel gear 920 may be positioned so the tilting head bevel gear 920 is perpendicular to the first driven bevel gear 919A and the second driven bevel gear 919B. The tilting head bevel gear 920 may be positioned so the tilting head bevel gear 920 interacts with the first driven bevel gear 919A and the second driven bevel gear 919B.

In some embodiments, a support member may be positioned between the first driven bevel gear 919A and the second driven bevel gear 919B. The support member may provide support to the first driven bevel gear 919A, the second driven bevel gear 919B, and/or the drive assembly 910 in order to increase a rigidity of the drive assembly 910.

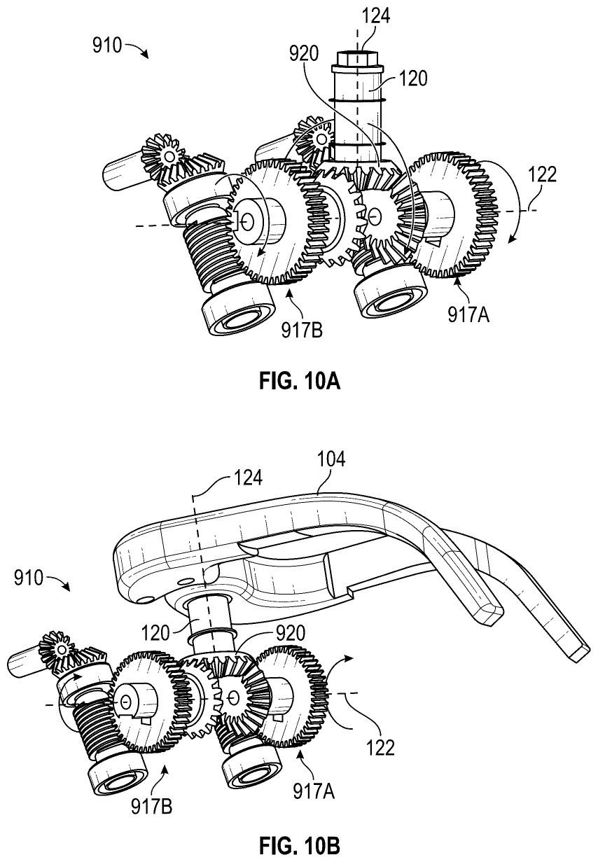

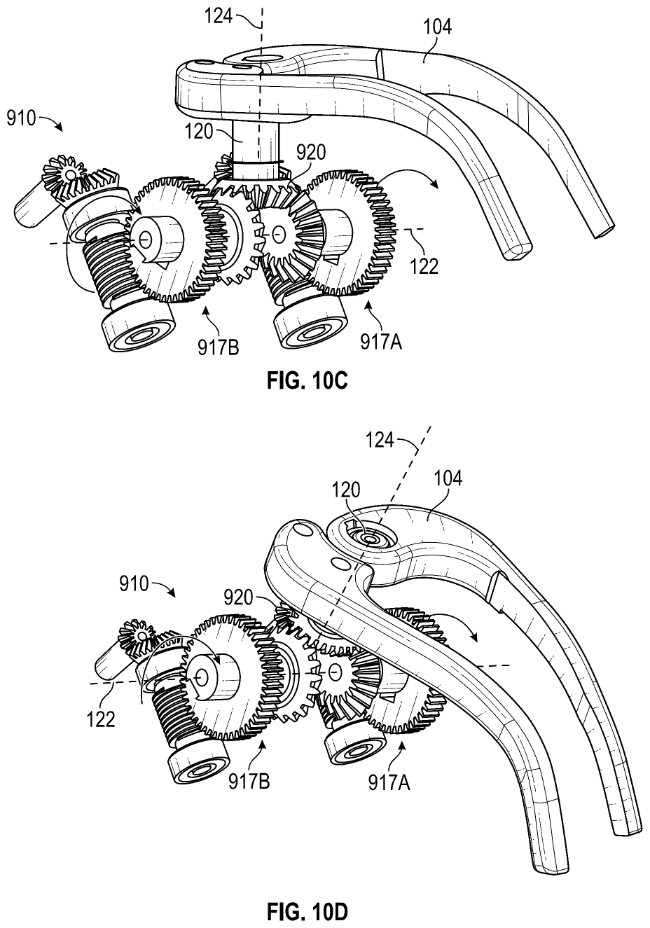

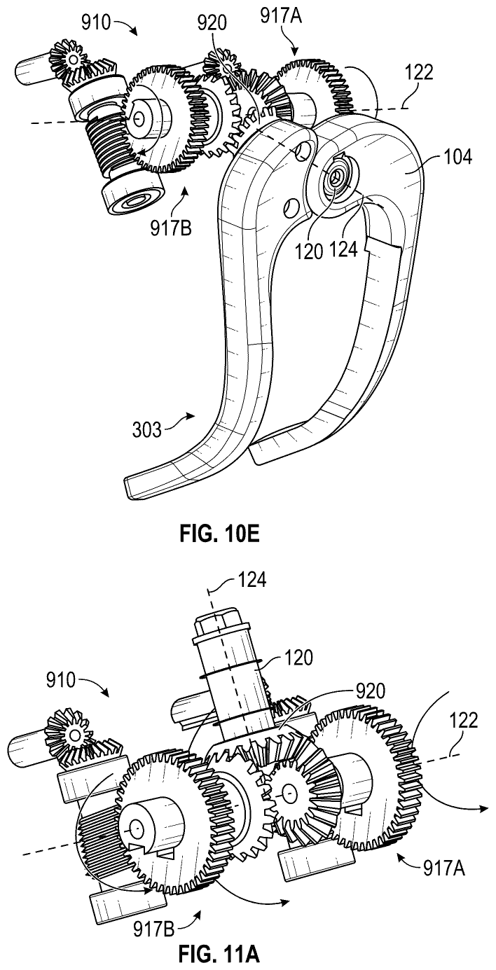

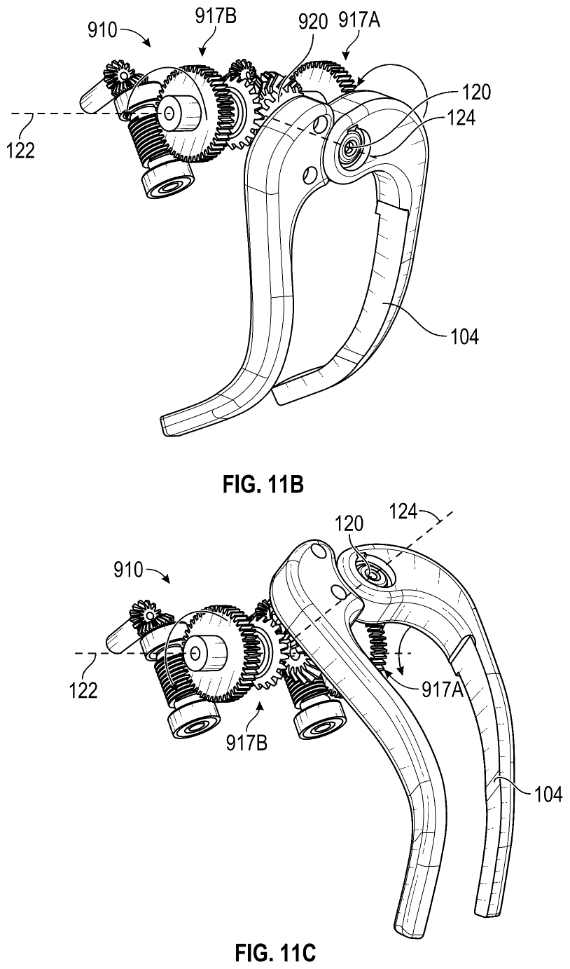

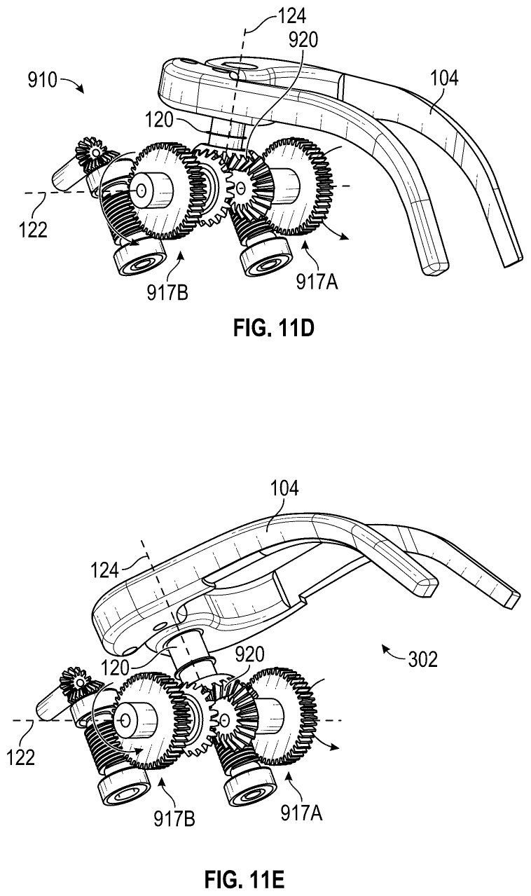

FIGS. 10A-10E and FIGS. 11A-11E illustrates the drive assembly 910 with the first driven gear assembly 917A and the second driven gear assembly 917B rotating in a same direction. When the first driven gear assembly 917A and the second driven gear assembly 917B rotate in the same direction at a same speed, the tilting head bevel gear 920, the tilting head 120, and/or the split hook 104 may rotate about the first axis 122. In some embodiments, the first driven gear assembly 917A and the second driven gear assembly 917B may rotate in the same direction at a same rotational speed in order to rotate the tilting head 120 about the first axis 122. When the first driven gear assembly 917A and the second driven gear assembly 917B rotate in the same direction at the same rotation speed, the first driven gear assembly 917A and the second driven gear assembly 917B may each apply a force to the tilting head bevel gear 920. The first driven gear assembly 917A and the second driven gear assembly 917B may apply the force in opposite rotational directions around the second axis 124. Accordingly, the force applied by the first driven gear assembly 917A and the force applied by the second driven gear assembly 917B may cancel each other out around the second axis 124 Therefore, the tilting head bevel gear 920 may not rotate about the second axis 124. The first driven gear assembly 917A and the second driven gear assembly 917B may apply the force in a same direction around the first axis 122. Accordingly, the force applied by the first driven gear assembly 917A and the second driven gear assembly 917B may rotate the tilting head bevel gear 920.

As shown in FIGS. 10B-10E, when the first driven gear assembly 917A and the second driven gear assembly 917B rotate in a first direction around the first axis 122, the tilting head bevel gear 920, the tilting head 120, and/or the split hook 104 may rotate in a direction towards the pinched position 303. As shown in FIGS. 11B-11E, when the first driven gear assembly 917A and the second driven gear assembly 917B rotate in a second direction around the first axis 122, the tilting head bevel gear 920, the tilting head 120, and/or the split hook 104 may rotate in a direction towards the extended position 302.

In some embodiments, the tilting head bevel gear 920 may include a mechanical stop. The mechanical stop may be engaged to prevent or inhibit rotation of the tilting head bevel gear 920 around the second axis 124.

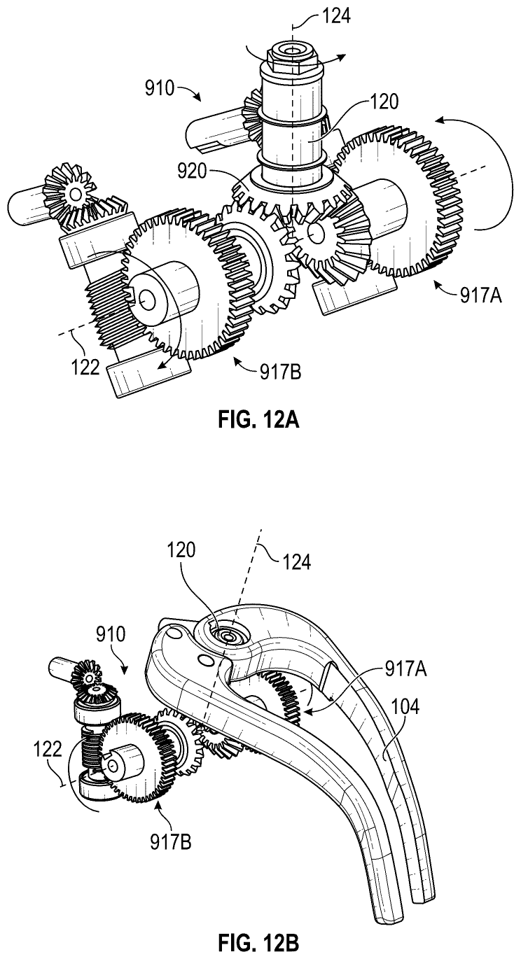

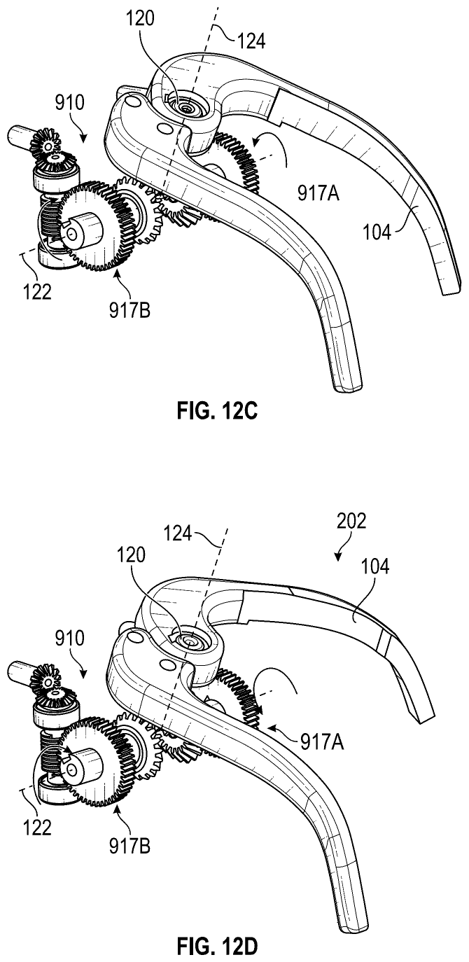

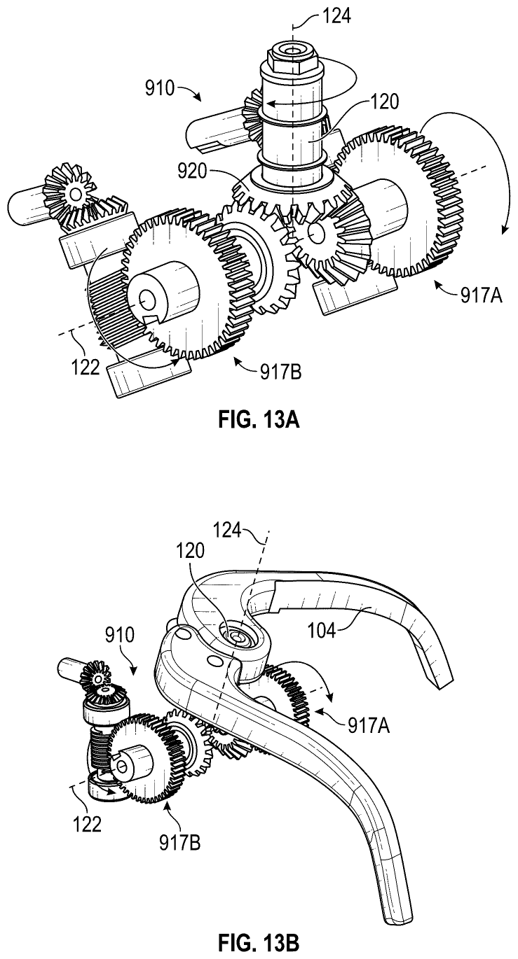

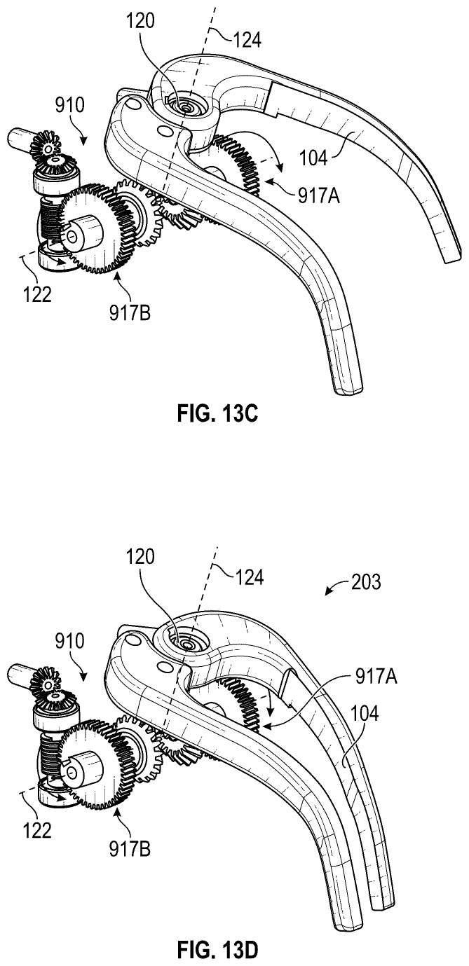

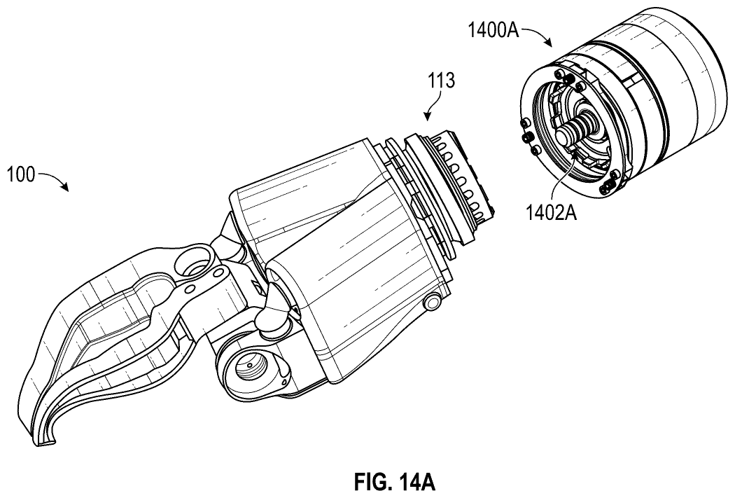

FIGS. 12A-12D and FIGS. 13A-13D illustrate the drive assembly 910 with the first driven gear assembly 917A and the second driven gear assembly 917B rotating in opposite directions relative to each other. In some embodiments, the first driven gear assembly 917A and the second driven gear assembly 917B may rotate in opposite direction at a same speed. In some embodiments, the first driven gear assembly 917A may rotate in a same direction as the second driven gear assembly 917B at twice the speed of the second driven gear assembly 917B. In some embodiments, the second driven gear assembly 917B may rotate in a same direction as the first driven gear assembly 917A at twice the speed of the first driven gear assembly 917A. When the first driven gear assembly 917A and the second driven gear assembly 917B rotate in opposite directions relative to each other, the tilting head bevel gear 920 may rotate about the second axis 124. When the first driven gear assembly 917A and the second driven gear assembly 917B rotate in opposite directions relative to each other, the first driven gear assembly 917A and the second driven gear assembly 917B may each apply a force to the tilting head bevel gear 920. The first driven gear assembly 917A and the second driven gear assembly 917B may apply the force in a same rotational direction around the second axis 124. Accordingly, the force applied by the first driven gear assembly 917A and the force applied by the second driven gear assembly 917B may cause the tilting head bevel gear 920 to rotate about the second axis 124. The first driven gear assembly 917A and the second driven gear assembly 917B may apply the force in opposite directions around the first axis 122. Accordingly, the force applied by the first driven gear assembly 917A and the force applied by the second driven gear assembly 917B may cancel each other out around the first axis 122. Therefore, the tilting head bevel gear 920 may not rotate about the first axis 122.

As shown in FIGS. 12B-12D, when the first driven gear assembly 917A rotates in the first direction around the first axis 122 and the second driven gear assembly 917B rotates in the second direction around the first axis 122, the tilting head bevel gear 920 and the tilting head 120 may rotate around the second axis 124 so the split hook 104 rotates towards the open position 202. As shown in FIGS. 13B-13D, when the first driven gear assembly 917A rotates in the second direction around the first axis 122 and the second driven gear assembly 917B rotates in the first direction around the first axis 122, the tilting head bevel gear 920 and the tilting head 120 may rotate around the second axis 124 so the split hook 104 rotates towards the closed position 203.

In some embodiments, the drive assembly 910 may rotate the tilting head bevel gear 920, the tilting head 120, and/or the split hook 104 around the first axis 122 and the second axis 124 at the same time. In some embodiments, the first driven gear assembly 917A and the second driven gear assembly 917B may be rotated in a same direction at different speeds in order to rotate the tilting head bevel gear 920, the tilting head 120, and/or the split hook 104 around the first axis 122 and the second axis 124 at the same time. In some embodiments, the first driven gear assembly 917A and the second driven gear assembly 917B may be rotated in opposite directions at different speeds in order to rotate the tilting head bevel gear 920, the tilting head 120, and/or the split hook 104 around the first axis 122 and the second axis 124 at the same time.

In some embodiments, the prosthetic device 100 may include one or more sensors configured to sense rotation of the motors 902 and/or one or more components of the drive assembly 910. In some embodiments, the one or more sensors may form an open control loop with a processor configured to control rotation of the motors 902.

It is to be appreciated that although the relative orientations and positions of components of the drive assembly 910 are described above, the relative orientations and positions of the components of the drive assembly 910 may be modified without departing from the scope of the disclosure. It is to be appreciated that although the gears of the drive assembly 910 are depicted with straight teeth, the gears of the drive assembly 910 may include spiral teeth, helical teeth, and/or any other type or shape of gear teeth. The end effectors 106, 108 of the split hook 104 may include any shape. For example, the end effectors 106, 108 may include a hook shape, a curved shape, a straight shape, a bent shape, and/or any other shape.

Further, in some embodiments, there may be more than one tilting head bevel gear 920, for example adjacent to each other. For example, the drive assembly 910 may include a first tilting head bevel gear 920 and a second tilting head bevel gear 920 coupled to the tilting head 120. The first end effector 106 may be coupled to the first tilting head bevel gear 920 and the second end effector 108 may be coupled to the second tilting head bevel gear 920. The first tilting head bevel gear 920 may be coupled to the driven gear assembly 917A and the second tilting head bevel gear 920 may be coupled to the driven gear assembly 917B. In some embodiments, an idler gear may be coupled to the first tilting head bevel gear 920 and the second tilting head bevel gear 920. The first driven gear assembly 917A and the second driven gear assembly 917B may rotate the tilting head 120 around the first axis 122 when the first driven gear assembly 917A and the second driven gear assembly 917B rotate at a same speed in the same direction around the first axis 122. The first driven gear assembly 917A and the second driven gear assembly 917B may rotate the first tilting head gear 920 and the second tilting head gear 920 in order to rotate the first end effector 106 and the second end effector 108 in opposite directions around the second axis 124 when the first driven gear assembly 917A and the second driven gear assembly 917B rotate at different speeds. Accordingly, both the first end effector 106 and the second end effector 108 may rotate around the second axis 124 in order to open and close split hook 104.

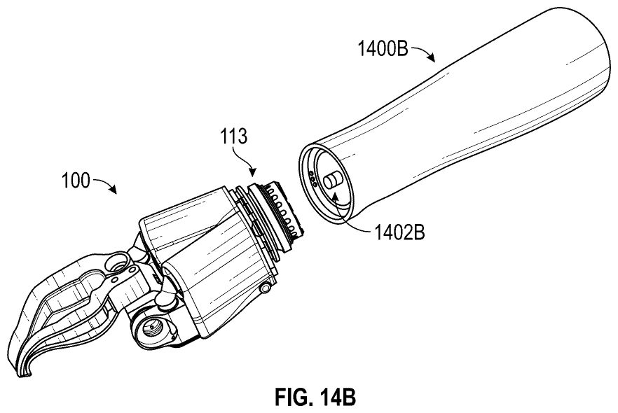

In some embodiments, the prosthetic device 100 may be configured to couple with, or be coupled with, a prosthetic wrist 1400A, as shown in FIG. 14A. In some embodiments, the prosthetic device 100 may be coupled to a prosthetic arm 1400B, as shown in FIG. 14B. In some embodiments, the connector 113 may couple the prosthetic device 100 to a connector 1402A, 1402B of the prosthetic wrist 1400A and the prosthetic arm 1400B. The prosthetic device 100 may couple with various prosthetic wrists and arms and other components, for example those wrists and arms and other components shown and described in U.S. Pat. No. 10,369,024 issued Aug. 6, 2019 and titled SYSTEMS AND METHODS FOR PROSTHETIC WRIST ROTATION, in U.S. Pat. No. 11,185,426 issued Nov. 30, 2021 and titled SYSTEMS AND METHODS FOR PROSTHETIC WRIST ROTATION, and in U.S. Pat. No. 10,449,063 issued Oct. 22, 2019 and titled WRIST DEVICE FOR A PROSTHETIC LIMB, the entire contents of each of which is incorporated by reference herein for all purposes and forms a part of this specification.

FIGS. 15-23 illustrate another example prosthetic assistive hand device or “prosthetic device” 1500 and features thereof. The prosthetic device 1500 may include any of the features of the prosthetic device 100, and vice versa, except as otherwise described herein. The prosthetic device 1500 provides numerous advantages such as a 3-jaw chuck, cylindrical grasping, and lateral grasping, with enhanced line of sight, versatility and ease of use. As further described herein, the prosthetic device 1500 may include a rotating plate underneath a chassis, allowing for multiple angles of thumb positioning (abduction-adduction) to provide versatile grasping capabilities. The ring may be manually rotated around a disc. A holding mechanism prevents the ring from rotating freely in absence of sufficient torque. This holding mechanism may be a ball detent mechanism that includes a spring-loaded ball within a circular channel of the disc. The holding mechanism may interact with integrated notches of the ring, for example seven notches, offering predefined positions. The thumb component may be directly integrated with the ring. Additionally, the thumb features flexion-extension, allowing it to be manually parked or opened. A spring-loaded ratchet mechanism may assist in engaging and disengaging the teeth as the thumb is manually adjusted to lock in the desired position. The mechanism may not be left- or right-handed, offering a unique size of device—(absence of right-handed, left-handed thumb feature.) However, merely for the sake of illustration of certain features, the terms “abducted,” “adducted,” “medial” and “lateral” and other directional terms may be used with reference to a right-handed arrangement. The plates may constrain the ring movement to one degree of freedom, for example rotation about a single axis. In some embodiments of the design, a boss or flange providing guidance to the ring can be part of either disc plate. The disc plates can each be single manufactured parts, an assembly of single manufactured parts or a feature of a larger manufactured part (e.g. main body of the device). The ratchet may provide more than one pre-defined positions for flexion/extension of the thumb, such as 5 pre-defined positions (in flexion-extension) and 7 positions (in adduction-abduction rotation). These and other features and advantages are described in further detail herein.

FIG. 15 is a perspective view of the prosthetic device 1500 having a thumb assembly 1550. The thumb assembly 1550 may be coupled to a proximal end 1503 of a palm portion 1502 of the prosthetic device 1500. The thumb assembly 1550 may include an opposable thumb (e.g., third end effector) 1510. The thumb assembly 1550 may include a rotation assembly 1552. A proximal (e.g., first) end 1516 of the opposable thumb 1510 may be coupled to the rotation assembly 1552. The rotation assembly 1552 may be coupled to the proximal end 1503 of the palm portion 1502 of the prosthetic device 1500. The rotation assembly 1552 may extend a distance 1512 forward from a palm wall 1501 of the palm portion 1502 so the proximal end 1516 of the opposable thumb 1510 is positioned the distance 1512 from the palm wall 1501. The rotation assembly 1552 may be configured to abduct and/or adduct the opposable thumb 1510. “Abduct” and “adduct” as used herein have their usual and customary meaning and include respectively, without limitation, moving a component away from and toward a midline or other geometric anatomical reference. The rotation assembly 1552 may be configured to rotate about a longitudinal axis 1524 of the prosthetic device 1500 in order to rotate the opposable thumb 1510 about the longitudinal axis 1524. The longitudinal axis 1524 may be an axis extending in a direction from the proximal end 1503 of the palm portion 1502 to a distal end 1505 of the palm portion 1502. The rotation assembly 1552 may define the longitudinal axis 1524. The longitudinal axis 1524 may be a centerline of a rotating, annular portion of the rotation assembly 1552.

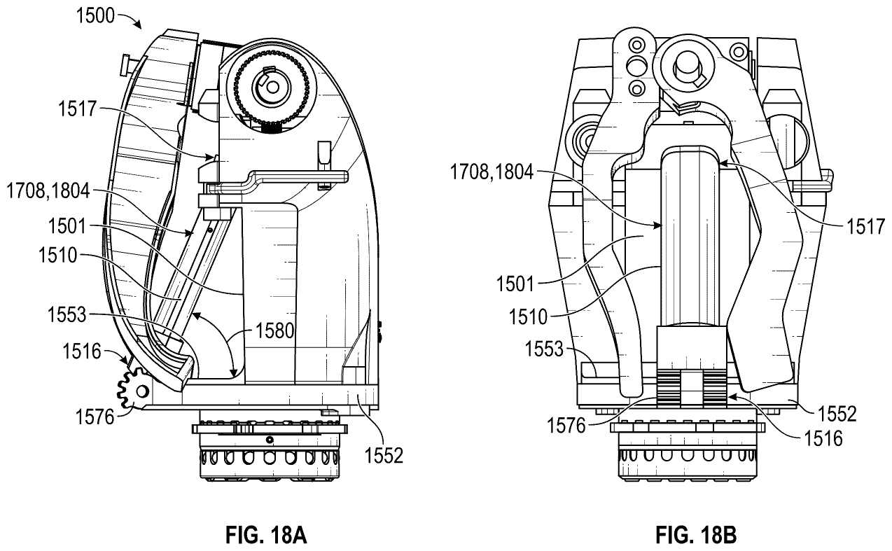

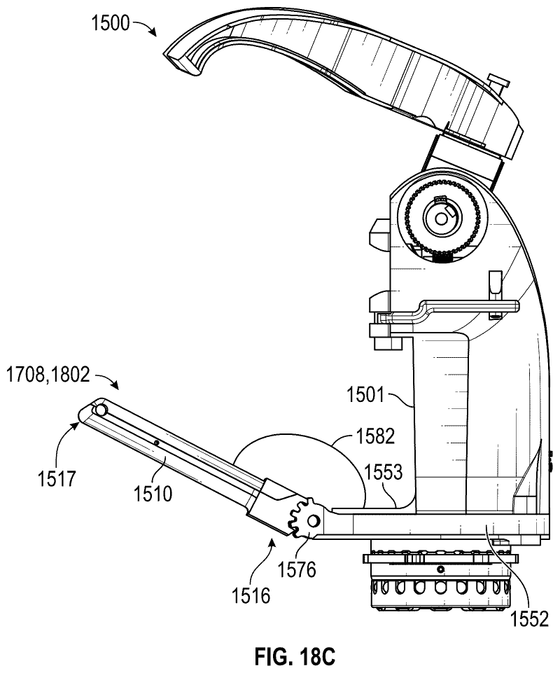

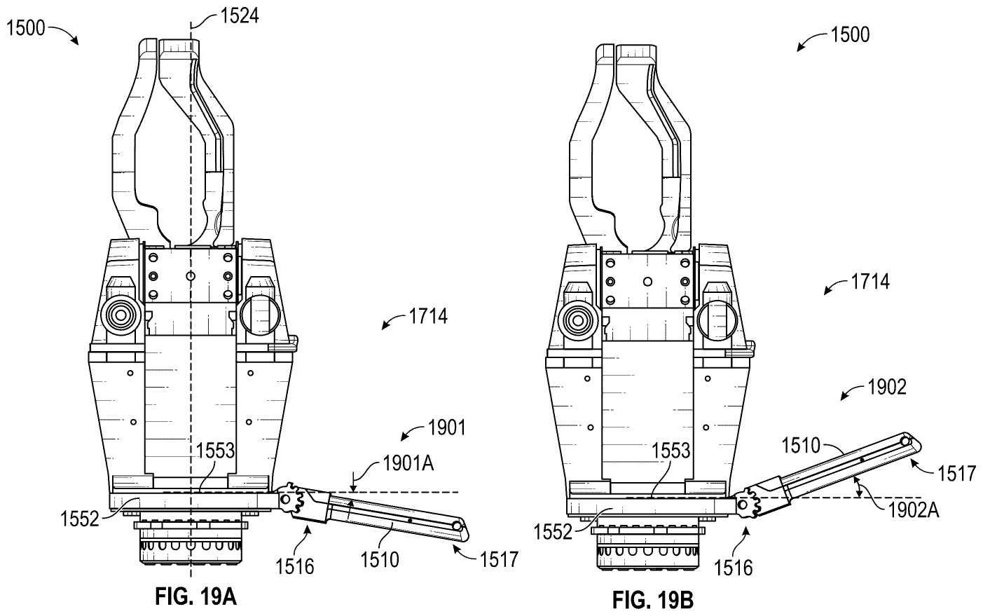

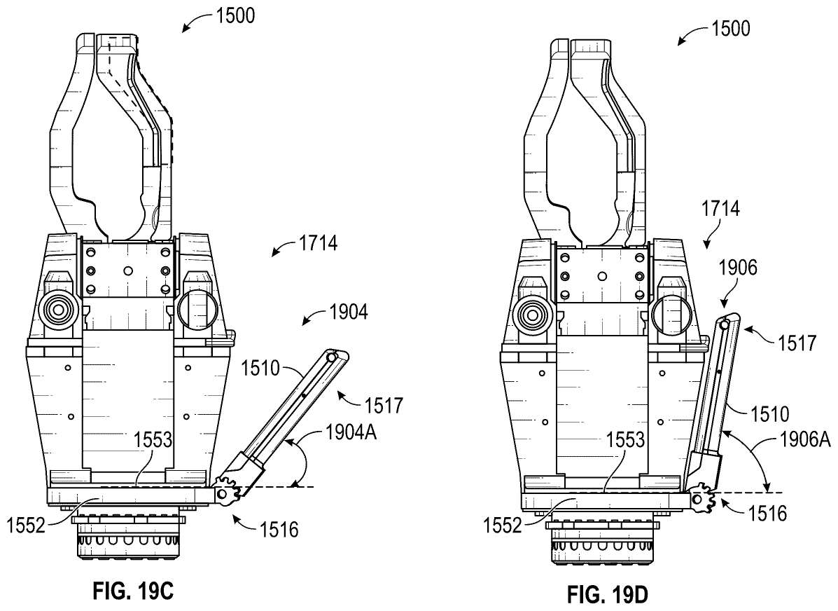

The proximal end 1516 of the opposable thumb 1510 may be rotatably coupled to the rotation assembly 1552. The opposable thumb 1510 may be configured to rotate about a transverse axis 1522. The transverse axis 1522 may be perpendicular to the longitudinal axis 1524. The transverse axis 1522 may extend through the prosthetic device 1500. The transverse axis 1522 may extend through the prosthetic device 1500 at the proximal end 1516 of the opposable thumb 1510. The transverse axis 1522 may correspond to a hinge line of the rotating opposable thumb 1510. The opposable thumb 1510 may be configured to rotate about the transverse axis 1522 between a fully extended position 1901, for example as shown in FIG. 19A, and a retracted position 1804, for example as shown in FIGS. 18A and 18B. The opposable thumb 1510 may rotate about the transverse axis 1522 in a pinching direction (toward the palm portion 1502) and an opposite, unpinching direction (away from the palm portion 1502).

The prosthetic device 1500 may include a release mechanism 1590. The release mechanism 1590 may be configured to release (e.g. disengage) a drive assembly 1592 of the prosthetic device 1500. The drive assembly 1592 may include any of the features of the drive assembly 910, and the drive assembly 910 may include any of the features of drive assembly 1592, except as otherwise described.

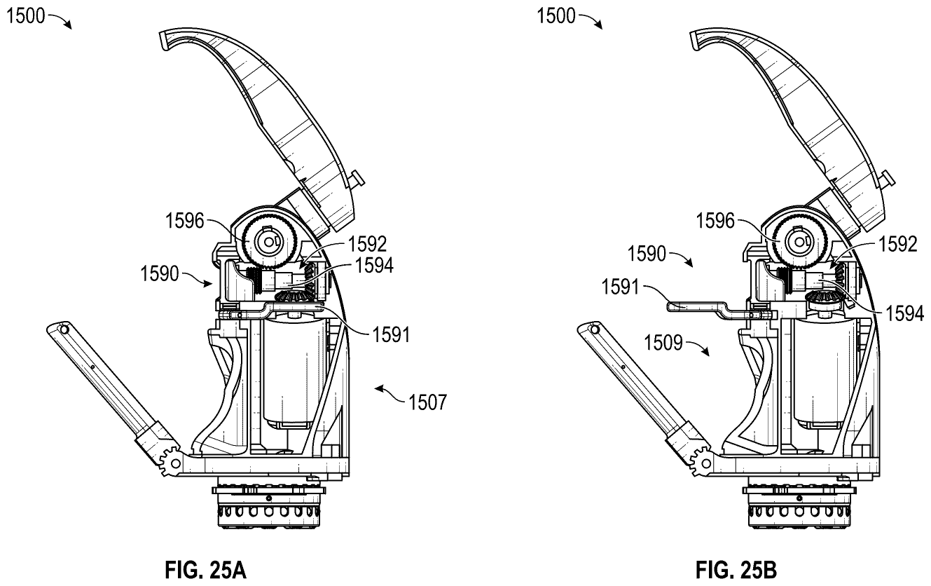

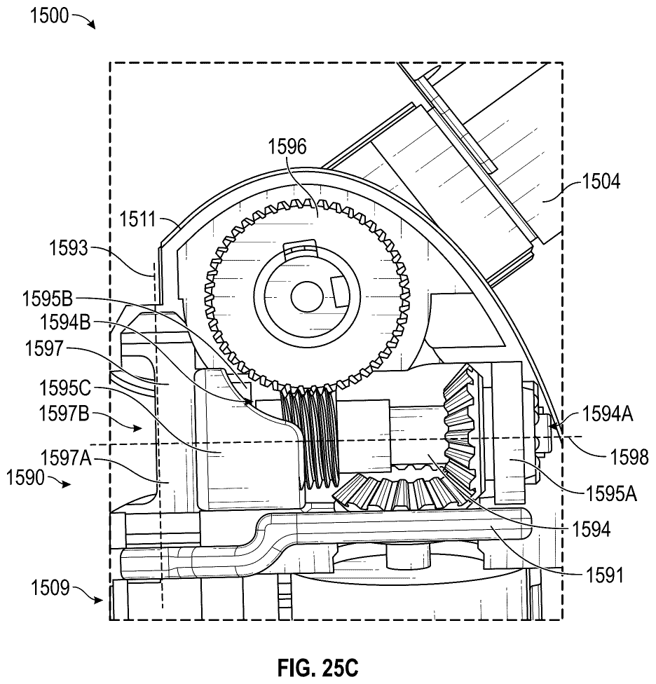

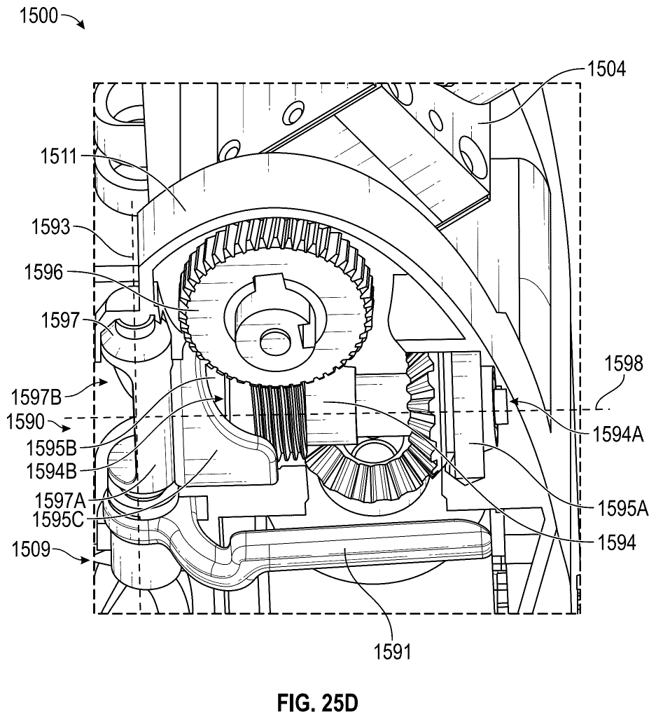

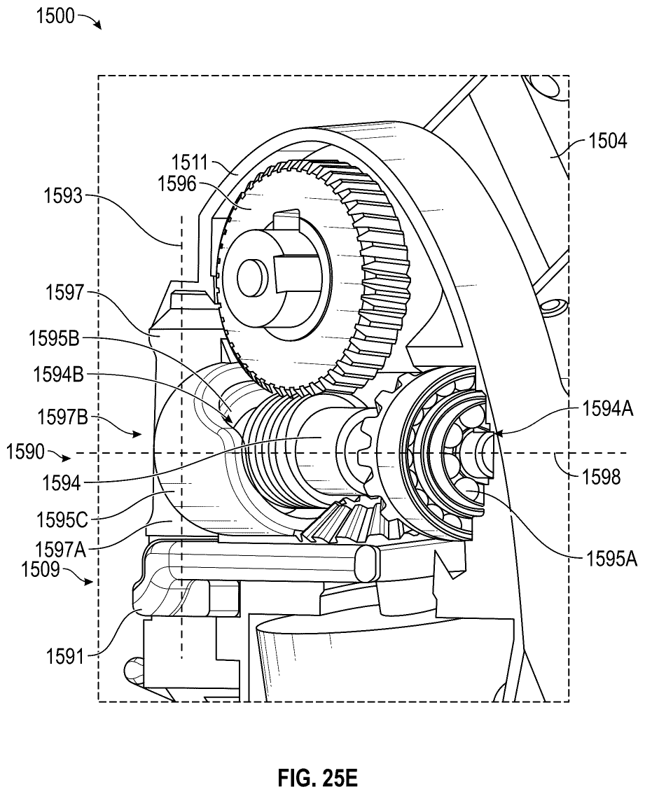

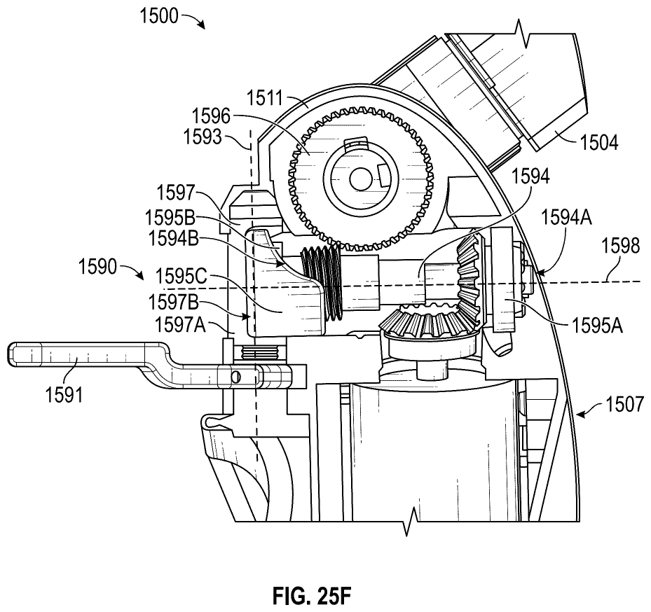

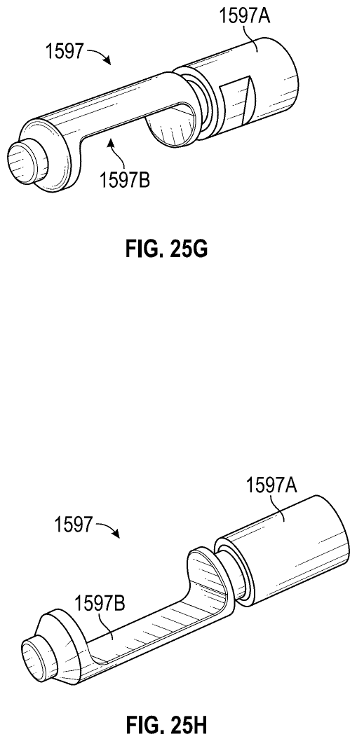

The release mechanism 1590 may be configured to release engagement and/or forces between gears of the release mechanism 1590. The release mechanism 1590 may be configured to disengage worm gears 1594 of the drive assembly 1592 from driven gears 1596 of the drive assembly 1592. The release mechanism 1590 may include a lever arm 1591 as shown which is rotated. The lever arm 1591 may be rotated about an axis 1593 that is parallel to the longitudinal axis 1524. The lever arm 1591 may rotate about an axis on the palm side 1509 of the device 1500. Rotation of the release mechanism 1590 may cause the gears to disengage. The release mechanism 1590 may be configured to translate the worm gears 1594 away from the driven gears 1596 so teeth of the worm gears 1594 and the driven gears 1596 do not interlock, as described in more detail with reference to FIGS. 25A-25H Accordingly, if the drive assembly 1592 is jammed, or when it is otherwise desired to release the gears, the release mechanism 1590 may be actuated to unjam or release the drive assembly 1592. When the release mechanism 1590 is actuated, a user may manually pinch/unpinch the split hook 1504 and/or open and close the split hook 1504. The lever arm 1591 may be rotated in the opposite manner to re-engage the gears.

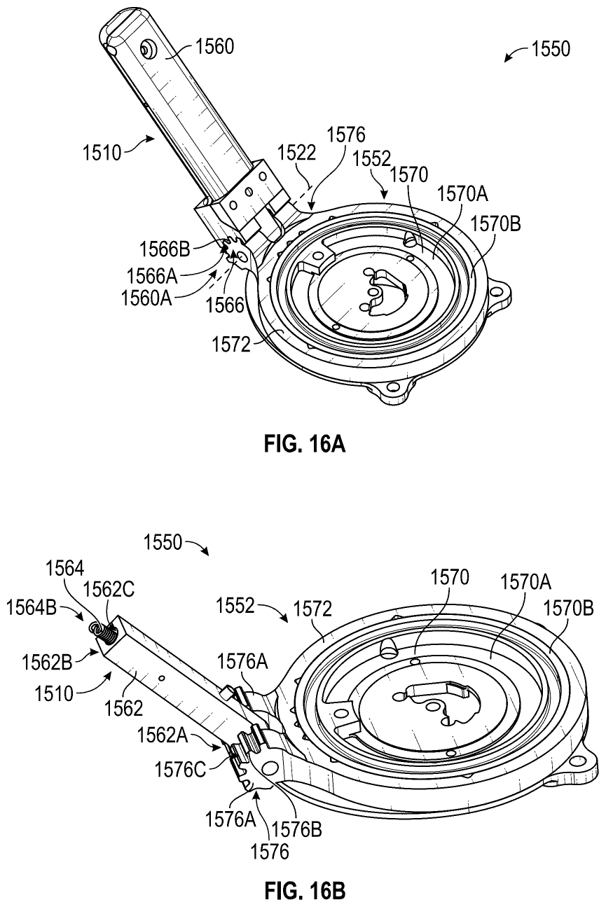

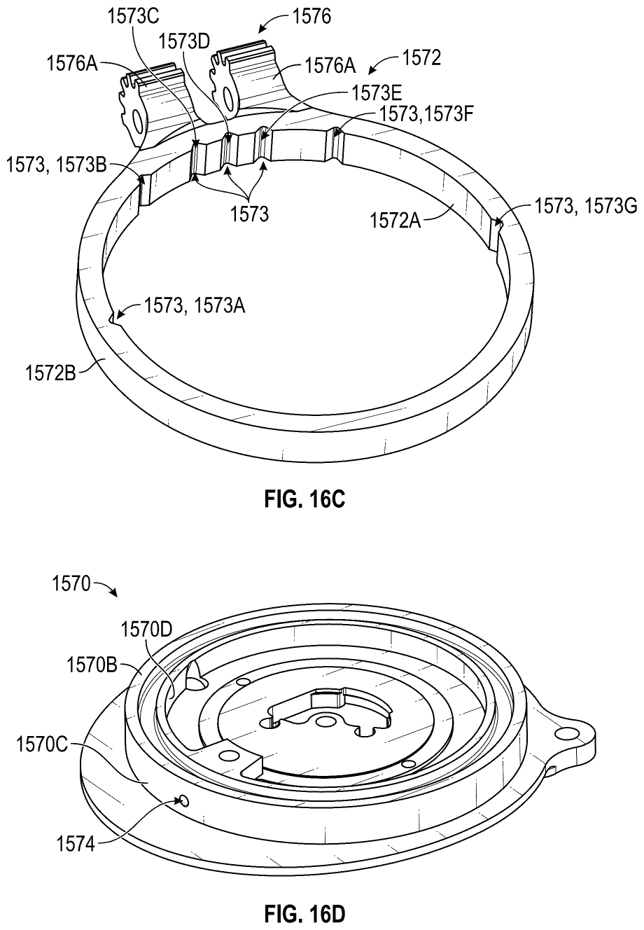

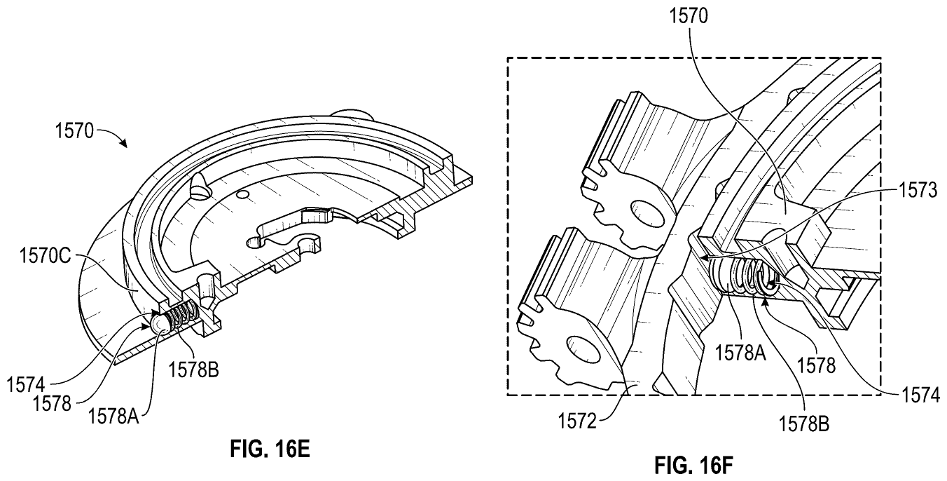

FIGS. 16A-16F illustrate the thumb assembly 1550 in isolation. FIG. 16A illustrates a perspective view of the thumb assembly 1550. FIG. 16B illustrates the thumb assembly 1550 with a cover 1560 of the opposable thumb 1510 removed. FIG. 16C illustrates a rotating member 1572 of the rotation assembly 1552 of the thumb assembly 1550 in isolation. FIG. 16D illustrates a plate 1570 of the rotation assembly 1552 of the thumb assembly 1550 in isolation. FIG. 16E illustrates a cross-section of the plate 1570 in isolation. FIG. 16F illustrates the rotation assembly 1552 with the plate 1570 shown in cross-section.

As shown in FIGS. 16A and 16B, the opposable thumb 1510 may include the cover 1560 and a body 1562 (shown in FIG. 16B). The cover 1560 may be positioned over the body 1562. The body 1562 may extend from a proximal end 1562A to a distal end 1562B. The cover 1560 may be slidably positioned over the body 1562. The cover 1560 may be configured to translate relative to the body 1562 towards and/or away from the proximal end 1562A of the body 1562 and towards and/or away from the distal end 1562B of the body 1562.

The body 1562 may include a channel 1562C. The channel 1562C may extend into the body 1562 from the distal end 1562B of the body 1562. The opposable thumb 1510 may include a biasing member 1564 positioned in the channel 1562C of the body 1562. A proximal end (not shown) of the biasing member 1564 may be coupled to the body 1562 at a proximal end of the channel 1562C. The biasing member 1564 may include a spring and/or any other suitable biasing member. A distal end 1564B of the biasing member 1564 may extend out of the channel 1562C of the body 1562. The distal end 1564B may be coupled to the cover 1560. The biasing member 1564 may bias the cover 1560 in the proximal direction so the cover 1560 is biased toward the proximal end 1562A of the body 1562. The biasing member 1564 may be a pull spring, such as extension or tension springs. The biasing member 1564 may include a close-wound coil spring with hooks on one or both ends.

The rotation assembly 1552 of the thumb assembly 1550 may include the plate 1570 and the rotating member 1572. The rotating member 1572 may be configured to rotate relative to the plate 1570. The rotating member 1572 may be annular, for example ring-shaped. The plate 1570 may include a base 1570A and a circular-shaped protrusion 1570B, such as a wall, extending axially from the base 1570A. The rotating member 1572 may be positioned circumferentially around the protrusion 1570B. The rotating member 1572 may be configured to rotate around the protrusion 1570B.

As shown in FIG. 16C, the rotating member 1572 may include a plurality of recesses 1573 extending radially into an inner surface 1572A of the rotating member 1572. The recesses 1573 may be elongated in the axial or longitudinal direction as shown. The recesses 1573 may extend axially in directions that are parallel to each other. The recesses 1573 may be circumferentially spaced around the rotating member 1572. The recesses 1573 may be circumferentially spaced around a portion of the rotating member 1572. In some embodiments, the recesses 1573 may be equally spaced around the rotating member 1572 or a portion of the rotating member 1572. In some embodiments, a distance between the recesses 1573 may vary. The rotating member 1572 may include two (2) recesses 1573, three (3) recesses 1573, four (4) recesses 1573, five (5) recesses 1573, six (6) recesses 1573, seven (7) recesses 1573, eight (8) recesses 1573, nine (9) recesses 1573, ten (10) recesses 1573, eleven (11) recesses 1573, twelve (12) recesses 1573, thirteen (13) recesses 1573, fourteen (14) recesses 1573, or fifteen (15) recesses 1573, or at least any of the aforementioned numbers of recesses 1573. In some embodiments, the rotating member 1572 may include more than fifteen (15) recesses 1573. The recesses 1573 may extend axially or longitudinally. The recesses 1573 may extend from an upper surface of the rotating member 1572 to a lower, opposite surface of the rotating member 1572, or any distance therebetween.

As shown in FIG. 16D, the plate 1570 may include a channel 1574 extending through the protrusion 1570B. The channel 1574 may extend radially through the protrusion 1570B from an outer surface 1570C of the protrusion 1570B to an inner surface 1570D of the protrusion 1570B. As shown in FIGS. 16E and 16F, a detent mechanism 1578 may be positioned in the channel 1574. The detent mechanism 1578 may include a ball and spring detent mechanism and/or any other suitable detent mechanism.

The detent mechanism 1578 may be configured to extend out of the channel 1574 and into one or more of the recesses 1573 when the one or more of the recesses 1573 is aligned with the channel 1574. The detent mechanism 1578 may be configured to prevent or inhibit rotation of the rotating member 1572 relative to the plate 1570. The detent mechanism 1578 may be configured to retract into the channel 1574 when a sufficiently high rotational force is applied to the rotating member 1572. The recesses 1573 may be sized and shaped such that when such a sufficiently high rotational force is applied to the rotating member 1572, the rotating member 1572 applies a force to the detent mechanism 1578 in order to cause the detent mechanism 1578 to retract into the channel 1574. The detent mechanism 1578 may be configured to at least partially extend into the recesses 1573 to thereby prevent rotation until the rotational force applied to the rotating member 1572 is larger than a force threshold. For example, when a first rotational force less than the force threshold is applied to the rotating member 1572, the force applied to the detent mechanism 1758 by the rotating member 1572 may cause the detent mechanism 1578 to partially retract the detent mechanism 1578 into the channel 1574, but the detent mechanism 1578 may remain extended into the recess 1573 so the detent mechanism 1578 prevents or inhibits rotation of the rotating member 1572. When a second rotational force greater than the force threshold is applied to the rotating member 1572, the force applied to the detent mechanism 1578 by the rotating member 1572 may cause the detent mechanism 1578 to retract into the channel 1574 and out of the recess 1573 so the detent mechanism 1578 does not prevent or inhibit rotation of the rotating member 1572. Accordingly, the recesses 1573 and the detent mechanism 1578 may allow the rotating member 1572 to rotate around the plate 1570 to predetermined positions, and the rotating member 1572 may remain at the predetermined positions until a rotational force greater than the force threshold is applied to the rotating member 1572. For rotational forces applied that are less than the force threshold, the rotating member 1572 may not rotate.

As shown in FIGS. 16E and 16F, the detent mechanism 1578 may include a ball 1578A and a spring 1578B. The ball 1578A and the spring 1578B may be positioned in the channel 1574. The spring 1578B may be configured to bias the ball 1578A radially outward from the channel 1574 past the outer surface 1570C and/or towards the rotating member 1572. Accordingly, as shown in FIG. 16F, the ball 1578A may extend into one of the recesses 1573 in the rotating member 1572 until a rotational force applied to the rotating member 1572 is large enough such that the rotating member 1572 applies a sufficient force to the ball 1578A, causing the spring 1578B to compress and the ball 1578A to retract into the channel 1574.

As shown in FIG. 16C, the rotating member 1572 may include a ratchet 1576. The ratchet 1576 may extend radially outwardly from an outer surface 1572B of the rotating member 1572. As shown in FIGS. 16A-16B, the opposable thumb 1510 may be rotatably coupled to the ratchet 1576. The proximal end 1562A of the body 1562 of the opposable thumb 1510 may be rotatably (e.g., hingedly) coupled to the ratchet 1576. The ratchet 1576 may include ratchet members 1576A. The proximal end 1562A of the body 1562 may be positioned between the ratchet members 1576A and rotatably coupled to the ratchet members 1576A.

The ratchet members 1576A may be gear shaped. The ratchet members 1576A may include a plurality of protrusions 1576B. The plurality of protrusions 1576B may be circumferentially spaced apart to form recesses 1576C between each protrusion 1576B. As shown in FIG. 16A, the cover 1560 of the opposable thumb 1510 may include ratchet protrusions 1566 at a proximal end 1560A of the cover 1560. The ratchet protrusions 1566 may include a plurality of protrusions 1566B spaced apart to form recesses 1566A between each protrusion 1566B.

As described above, the opposable thumb 1510 may rotate about the transverse axis 1522 between an extended position 1802, as shown in FIG. 18C, and a retracted position 1804, as shown in FIGS. 18A and 18B. The rachet members 1576A and the ratchet protrusions 1566 may interlock to maintain the opposable thumb 1510 at discrete positions between the extended position 1802 and the retracted position 1804. The ratchet protrusions 1566 of the cover 1560 may be aligned with the ratchet members 1576A of the ratchet 1576 so the ratchet members 1576A extend into the ratchet protrusions 1566 of the cover 1560. The ratchet member 1576A and the ratchet protrusions 1566 may be configured to interlock to prevent or inhibit rotation of the opposable thumb 1510 (e.g., the cover 1560 and the body 1562) about the transverse axis 1522 relative to the rotating member 1572. The protrusions 1576B of the ratchet members 1576A may extend into the recesses 1566A of the ratchet protrusions 1566 of the cover 1560, and the protrusions 1566B between each recess 1566A of the ratchet protrusions 1566 may extend into the recesses 1576C of the ratchet members 1576A.

As described above, the biasing member 1564 may proximally bias the cover 1560 toward the proximal end 1652A of the body 1562. Accordingly, the biasing member 1564 may proximally bias the cover 1560 toward the ratchet members 1576A. The biasing member 1564 may prevent or inhibit the cover 1560 from moving away from the ratchet members 1576A, to thereby prevent or inhibit the ratchet protrusions 1566 from disengaging with the ratchet members 1576A, until a force applied to the cover 1560 in a distal direction away from the ratchet members 1576A is greater than the proximal force applied to the cover 1560 by the biasing member 1564. The cover 1560 may be manually moved distally, for example by a user of the device. When the distal force applied to the cover 1560 greater than the proximal force applied to the cover 1560 by the biasing member 1564, the cover 1560 may move (e.g. translate) in a distal direction away from the ratchet members 1576A. Accordingly, the ratchet protrusions 1566 may disengage from the ratchet members 1576A, and the opposable thumb 1510 may then rotate about the transverse axis 1522 relative to the rotating member 1572. The opposable thumb 1510 may be manually rotated, for example by the user.

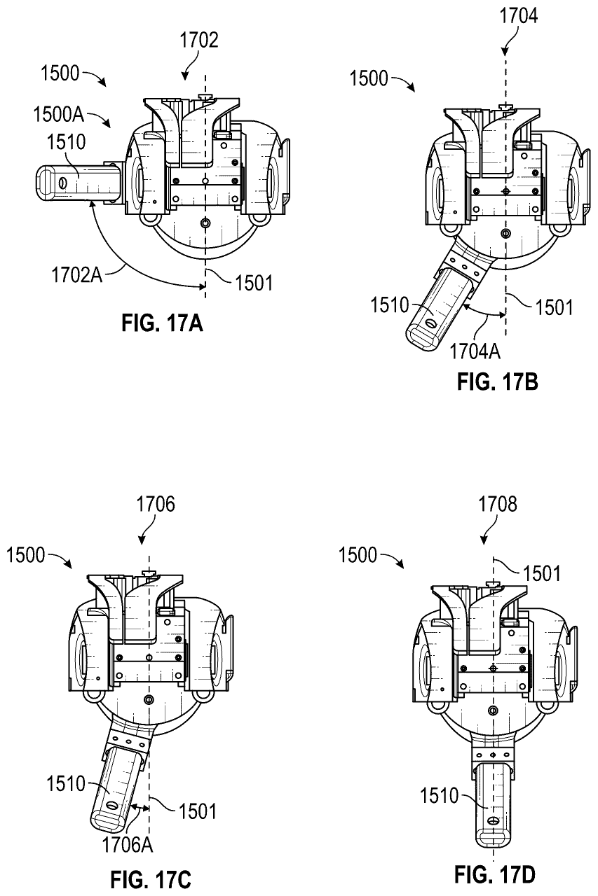

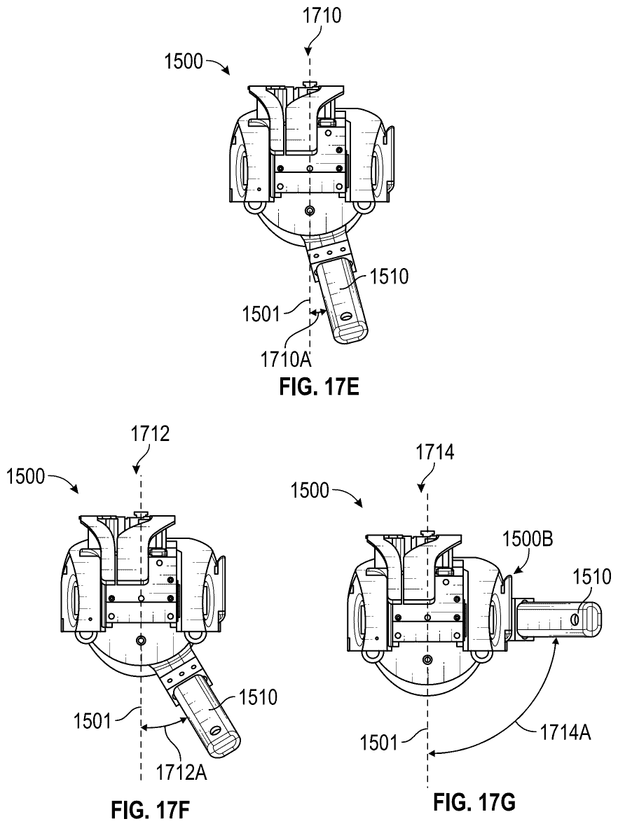

FIGS. 17A-17G illustrate sequential rotations of the opposable thumb 1510 about the longitudinal axis 1524. FIG. 17A illustrates the prosthetic device 1500 with the opposable thumb 1510 in an abducted position 1702 (or “frontal-external” position). FIG. 17B illustrates the prosthetic device 1500 with the opposable thumb 1510 in a first partially abducted position 1704 (or “mid-sagittal external” position). FIG. 17C illustrates the prosthetic device 1500 with the opposable thumb 1510 in a second partially abducted position 1706 (or “para-sagittal external” position). FIG. 17D illustrates the prosthetic device 1500 with the opposable thumb 1510 in a middle position 1708 (or “sagittal” position). FIG. 17E illustrates the prosthetic device 1500 with the opposable thumb 1510 in a first partially adducted position 1710 (or “para-sagittal internal” position). FIG. 17F illustrates the prosthetic device in a second partially adducted position 1712 (or “mid-sagittal internal” position). FIG. 17G illustrates the prosthetic device 1500 with the opposable thumb 1510 in an adducted position 1714 (or “frontal-internal” position).

As shown in FIG. 17A, in the abducted position 1702, the rotation assembly 1552 may be configured such that the opposable thumb 1510 is positioned on a first (lateral) side 1500A of the prosthetic device 1500. In the abducted position 1702, the opposable thumb 1510 may extend in a lateral direction. The opposable thumb 1510 (e.g. an axis along which the opposable thumb 1510 extends) may extend at an angle 1702A from a sagittal axis 1701. The sagittal axis 1701 may be an axis extending perpendicular to the longitudinal axis 1524 in the sagittal plane. The sagittal axis may intersect a dorsal side 1507 and a palm side 1509 of the prosthetic device 1500. As oriented in the figures, the angle 1702A may be measured relative to a portion of the sagittal axis 1701 that extends out from the dorsal side 1507. In some embodiments, the angle 1702A may be from 75 degrees to 105 degrees, from 80 degrees to 100 degrees, from 85 degrees to 95 degrees, from 88 degrees to 92 degrees, 90 degrees, or about 90 degrees. When the opposable thumb 1510 is in the abducted position 1702, a first recess 1573A (shown in FIG. 16C) of the plurality of recesses 1573 in the rotating member 1572 may be aligned with the channel 1574. The opposable thumb 1510 may be fully extended in this configuration (functionally parked) and thus easily reachable and can be rotated without the use of the contralateral hand, which may help a bilateral user or a unilateral user with an occupied contralateral hand.

As shown in FIG. 17B, in the first partially abducted position 1704, the rotation assembly 1552 may be configured such that the opposable thumb 1510 extends laterally at an angle 1704A from the sagittal axis 1701. The angle 1704A may be an angle less than the angle 1702A. In some embodiments, the angle 1704A may be less than 90 degrees. In some embodiments, the angle 1704A may be less than 75 degrees. In some embodiments, the angle 1704A may be from 15 degrees to 45 degrees, from 25 degrees to 35 degrees, from 28 degrees to 32 degrees, 30 degrees, or about 30 degrees. When the opposable thumb 1510 is in the first partially abducted position 1704, a second recess 1573B (shown in FIG. 16C) of the recesses 1573 in the rotating member 1572 may be aligned with the channel 1574. The second recess 1573B may be circumferentially spaced from the first recess 1573A in a first circumferential direction. With the opposable thumb 1510 in this rotational position and partially extended, the opposable thumb 1510 may provide an equal repartition of the pressure on an object being grasped, with the thumb opposite the open split hook approximatively on the center, offering a more stable grasp of the object.

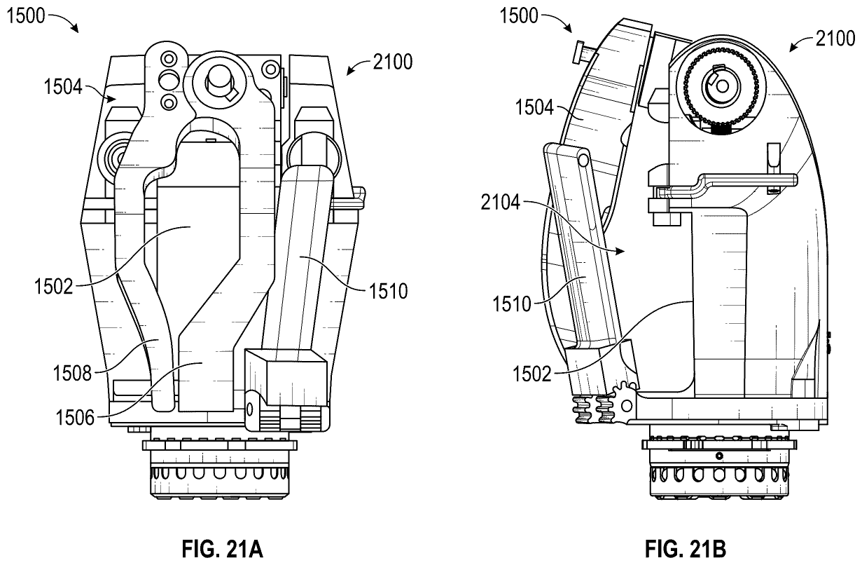

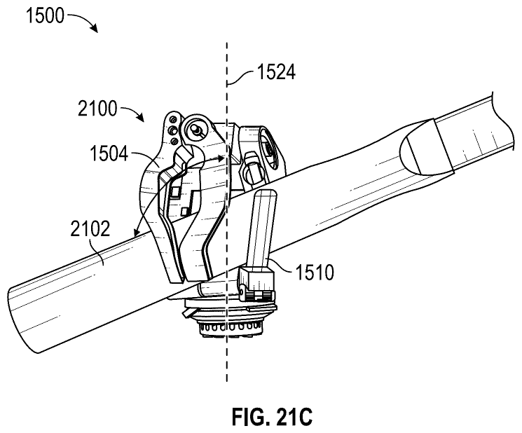

As shown in FIG. 17C, in the second partially abducted position 1706, the rotation assembly 1552 may be configured such that the opposable thumb 1510 extends laterally at the angle 1706C from the sagittal axis 1701. The angle 1706C may be an angle less than the angle 1704A. In some embodiments, the angle 1706C may be less than 45 degrees, less than 40 degrees, less than 30 degrees, from 5 degrees to 30 degrees, from 5 degrees to 15 degrees, from 8 degrees to 12 degrees, 10 degrees, or about 10 degrees. When the opposable thumb 1510 is in the second partially abducted position 1706, a third recess 1573C (shown in FIG. 16C) of the plurality of recesses 1573 in the rotating member 1572 may be aligned with the channel 1574. The third recess 1573C may be circumferentially spaced from the second recess 1573B in the first circumferential direction. In this rotational position, and with the opposable thumb 1510 partially extended, the thumb can provide for precise pinch for a right or left configuration to match with either split hook configuration. Further, such orientation may provide the capability to hold a tool handle with a natural 100 to 110 degree tilt to the tool that is recommended to operate manual tools, which can assist users with being productive and possibly returning to work. The position may also offer a better line of sight for screen use. Further details of such grip are shown in and described with respect to FIG. 21C.

As shown in FIG. 17D, in the middle position 1708, the rotation assembly 1552 may be configured such that the opposable thumb 1510 extends along the sagittal axis 1701. In some embodiments, there may be a slight angle between the opposable thumb 1510 and the sagittal axis, for example no more than 5 degrees, or no more than 3 degrees. When the opposable thumb is in the middle position 1708, a fourth recess 1573D (shown in FIG. 16C) of the plurality of recesses 1573 in the rotating member 1572 may be aligned with the channel 1574. The fourth recess 1573D may be circumferentially spaced from the third recess 1573C in the first circumferential direction. In this rotational position, the opposable thumb 1510 may be fully “parked” via full flexion in the sagittal plane toward the palm portion, hiding fully the opposable thumb 1510. This may offer a fist-like discrete option, which may provide the user with more comfort aesthetically in social settings, and provide an ease of functional activity of daily life like sliding a sleeve. With the thumb rotated outward (extended), the opposable thumb 1510 may offer a direct opposition with the split hook offering a pinch grip. When using a 3-jaw chuck grasp, the cylindrical grasp can hold an object perpendicular (for example, a glass of water can be held without tilting).

As shown in FIG. 17E, in the first partially adducted position 1710, the rotation assembly 1552 may be configured such that the opposable thumb 1510 extends at an angle 1710A from the sagittal axis 1701. In some embodiments, the opposable thumb 1510 may extend medially (e.g., towards a midline of a user's body) at the angle 1710A. The angle 1710A may have any of the values as described with respect to the angle 1706A, but in the opposite direction. When the opposable thumb 1510 is in the first partially adducted position 1710, a fifth recess 1573E (shown in FIG. 16C) of the plurality of recesses 1573 in the rotating member 1572 may be aligned with the channel 1574. The fifth recess 1573E may be circumferentially spaced from the fourth recess 1573D in the first circumferential direction. In this rotational position, and with the opposable thumb 1510 partially extended, the thumb may provide similar advantages as described with respect to FIG. 17C.

As shown in FIG. 17F, in the second partially adducted position 1712, the rotation assembly 1552 may be configured such that the opposable thumb 1510 extends medially at an angle 1712A from the sagittal axis 1701. The angle 1712A may have any of the values as described with respect to the angle 1704A, but in the opposite direction. In some embodiments, the angle 1712A may be an angle larger than the angle 1710A. When the opposable thumb 1510 is in the second partially adducted position 1712, a sixth recess 1573F (shown in FIG. 16C) of the recesses 1573 in the rotating member 1572 may be aligned with the channel 1574. The sixth recess 1573F may be circumferentially spaced from the fifth recess 1573E in the first circumferential direction. With the opposable thumb 1510 in this rotational position and partially extended, the opposable thumb 1510 may provide similar advantages as described with respect to FIG. 17B.

As shown in FIG. 17G, in the adducted position 1714, the rotation assembly 1552 may be configured such that the opposable thumb 1510 is positioned on a second (medial) side 1500B of the prosthetic device 1500 that is opposite the first (lateral) side 1500A. In the adducted position 1714, the opposable thumb 1510 may extended in a medial direction. The opposable thumb 1510 may extend at an angle 1714A from the sagittal axis 1701. In some embodiments, the angle 1714A may have any of the values as described with respect to the angle 1702A, but in the opposite direction. In some embodiments, the angle 1714A may be larger than the angle 1712A. When the opposable thumb 1510 is in the adducted position 1714, a seventh recess 1573G (shown in FIG. 16C) of the plurality of recesses 1573 in the rotating member 1572 may be aligned with the channel 1574. The seventh recess 1573G may be circumferentially spaced from the sixth recess 1573F in the first circumferential direction. With the opposable thumb 1510 fully parked (full flexion) in this rotational position, the opposable thumb 1510 may provide various advantages, as described with respect to FIG. 17A.

The various recesses may be located to provide a desired rotational position. As shown in FIG. 16C, the second recess 1573B may be positioned between the first recess 1573A and the third recess 1573C. The third recess 1573C may be positioned between the second recess 1573B and the fourth recess 1573D. The fourth recess 1573D may be positioned between the third recess 1573C and the fifth recess 1573E. The fourth recess 1573D may be positioned on the rotating member 1572 so the fourth recess 1573D is aligned with the ratchet 1576. The fifth recess 1573E may be positioned between the fourth recess 1573D and the sixth recess 1573F. The sixth recess 1573F may be positioned between the fifth recess 1573E and the seventh recess 1573G. The recesses 1573A, 1573B, 1573C, 1573D, 1573E, 1573F, 1573G may be positioned and/or spaced apart on the inner surface 1572A of the rotating member 1572 at corresponding angular positions along the inner surface 1572A as measured about the longitudinal axis 1524, so that the opposable thumb 1510 extends at the angles 1702A, 1704A, 1706A, 1708A, 1710A, 1712A, 1714A when channel 1574 is aligned with the corresponding recess 1573A, 1573B, 1573C, 1573D, 1573E, 1573F, 1573G.

FIGS. 18A and 18B illustrate the prosthetic device 1500 with the rotatable assembly in the middle position 1708 and the opposable thumb 1510 in the retracted position 1804. In the retracted positioned 1804, the opposable thumb 1510 may extend from the ratchet 1576 in a direction toward the palm wall 1501 of the prosthetic device 1500. Accordingly, the proximal end 1516 of the opposable thumb 1510 may be positioned farther from the palm wall 1501 than a distal end 1517 of the opposable thumb 1510, or in other words, the distal end 1517 of the opposable thumb 1510 may be positioned closer to the palm wall 1501 than the proximal end 1516. The opposable thumb 1510 may contact the palm wall 1501 when the opposable thumb 1510 is in the retracted position 1804. In the retracted position, the opposable thumb 1510 may extend from the ratchet 1576 at an angle 1580 with a plane perpendicular to the longitudinal axis 1524 (for example with a top surface 1553 of the rotation assembly 1552 as shown). The angle 1580 may be less than 90 degrees.

FIG. 18C illustrates the prosthetic device 1500 with the opposable thumb 1510 rotated about the longitudinal axis to the middle position 1708 and rotated about the transverse axis to the extended position 1802. In the extended position 1802, the opposable thumb 1510 may extend from the ratchet 1576 in a direction away from the palm wall 1501 of the prosthetic device 1500. Accordingly, the proximal end 1516 of the opposable thumb 1510 may be positioned closer to the palm wall 1501 than the distal end 1517 of the opposable thumb 1510, or in other words, the distal end 1517 may be positioned farther from the palm wall 1501 than the proximal end 1516 of the opposable thumb 1510. In the extended position, the opposable thumb 1510 may extend from the ratchet 1576 at an angle 1582 with a plane that is perpendicular to the longitudinal axis 1524 (for example, with the top surface 1553 of the rotation assembly 1552). The angle 1582 may be greater than 90 degrees, greater than 135 degrees, greater than 150 degrees, from 90 degrees to 180 degrees, from 135 degrees to 180 degrees, from 150 degrees to 180 degrees, or about 160 degrees.

FIG. 19A-19D illustrate the opposable thumb 1510 in the adducted position 1714 in various rotational positions about the transverse axis 1522. FIG. 19A illustrates the prosthetic device 1500 with the opposable thumb 1510 in a fully extended position 1901. FIG. 19B illustrates the prosthetic device 1500 with the opposable thumb 1510 in a first partially extended position 1902. FIG. 19C illustrates the prosthetic device 1500 with the opposable thumb 1510 in a second partially extended position 1904. FIG. 19D illustrates the prosthetic device 1500 with the opposable thumb 1510 in a third partially extended position 1906.

As shown in FIG. 19A, when the opposable thumb 1510 is in the fully extended position 1901, the opposable thumb 1510 may extend proximally from the rotation assembly 1552. The distal end 1517 of the opposable thumb 1510 may therefore be positioned lower (along the direction of the longitudinal axis 1524) than the proximal end 1516 of the opposable thumb 1510 as oriented in the figure. The proximal end 1516 may be vertically closer, along the longitudinal axis 1524, to a distal end of the assistive device as compared to the distal end 1517. The opposable thumb 1510 (for example an axis along which the opposable thumb 1510 extends) may extend from the rotation assembly 1552 at an angle 1901A with a plane that is perpendicular to the longitudinal axis 1524 (for example, with the top surface 1553 of the rotation assembly 1552 as shown). In some embodiments, the angle 1901A may be less than 45 degrees, less than 30 degrees, less than 15 degrees, from 5 degrees to 15 degrees, from 8 degrees and 12 degrees, 10 degrees, or about 10 degrees. In some embodiments, the opposable thumb 1510 may extend parallel with the top surface 1553 of the rotation assembly 1552 so the opposable thumb 1510 extends perpendicular to the longitudinal axis 1524. Accordingly, in some embodiments, the angle 1901A may be about 0 degrees.

As shown in FIG. 19B, when the opposable thumb 1510 is in the first partially extended position 1902, the opposable thumb 1510 may extend distally from the rotation assembly 1552. In some embodiments, the first partially extended position 1902 may be a same position as the extended position 1802 shown in FIG. 18C. The distal end 1517 of the opposable thumb 1510 may be positioned higher (along the longitudinal axis 1524) than the proximal end 1516 of the opposable thumb 1510 as oriented in the figure. The distal end 1517 may be vertically closer, along the longitudinal axis 1524, to a distal end of the assistive device as compared to the proximal end 1516. The opposable thumb 1510 may extend from the rotation assembly 1552 at an angle 1902A with a plane that is perpendicular to the longitudinal axis 1524 (for example, with the top surface 1553 of the rotation assembly 1552). In some embodiments, the angle 1902A may be less than 45 degrees, less than 30 degrees, from 10 degrees to 30 degrees, from 15 degrees to 25 degrees, from 18 degrees to 22 degrees, 20 degrees, or about 20 degrees.

As shown in FIG. 19C, when the opposable thumb 1510 is in the second partially extended position 1904, the opposable thumb 1510 may extend distally from the rotation assembly 1552. The distal end 1517 of the opposable thumb 1510 may be positioned higher (along the longitudinal axis 1524) than the proximal end 1516 of the opposable thumb 1510 as oriented in the figure. The distal end 1517 may be vertically closer, along the longitudinal axis 1524, to a distal end of the assistive device as compared to the proximal end 1516. The opposable thumb 1510 may extend from the rotation assembly 1552 at an angle 1904A with a plane that is perpendicular to the longitudinal axis 1524 (for example, with the top surface 1553 of the rotation assembly 1552). The angle 1904A may be an angle larger than the angle 1902A. In some embodiments, the angle 1904A may be less than 90 degrees, from 15 degrees to 90 degrees, from 30 degrees to 90 degrees, from 35 degrees to 65 degrees, from 45 degrees to 55 degrees, from 48 degrees to 52 degrees, 50 degrees, or about 50 degrees.

As shown in FIG. 19D, when the opposable thumb 1510 is in the third partially extended position 1906, the opposable thumb 1510 may be adjacent to and/or contact the palm portion 1502 of the prosthetic device. The opposable thumb 1510 may extend parallel with a sidewall 1511A of a housing 1511 of the prosthetic device 1500. When the opposable thumb 1510 is in the third partially extended position 1906, at least a portion of the opposable thumb 1510 may contact the sidewall 1511A. The opposable thumb 1510 may extend from the rotation assembly 1552 at an angle 1906A with the top surface 1553 of the rotation assembly 1552. The angle 1906A may be an angle larger than the angle 1904A.

Although the opposable thumb 1510 is shown in FIGS. 18A-18C and FIGS. 19A-19D as positioned in particular positions (for example, in the extended position 1802, the retracted position 1804, the fully extended position 1901, the first partially extended position 1902, the second partially extended position 1904, and the third partially extended position 1906), the opposable thumb 1510 may rotate about the transverse axis 1522 to one or more discrete positions between any of these positions. The number of discrete positions to which the opposable thumb 1510 rotates may be based on the number of the recesses 1566A and/or protrusions 1566B of the ratchet protrusions 1566, and/or the number of protrusions 1576B and/or recesses 1576C of the ratchet members 1576A. Further, the opposable thumb 1510 may rotate about the transverse axis 1522 while in any of the rotational positions relative to the longitudinal axis 1524, and vice versa. Thus, the opposable thumb 1510 may have any of the rotational abduction/adduction positions shown in or described with respect to FIGS. 17A to 17G in combination with any of the rotational pinching/unpinching positions shown in or described with respect to FIGS. 19A-19D.

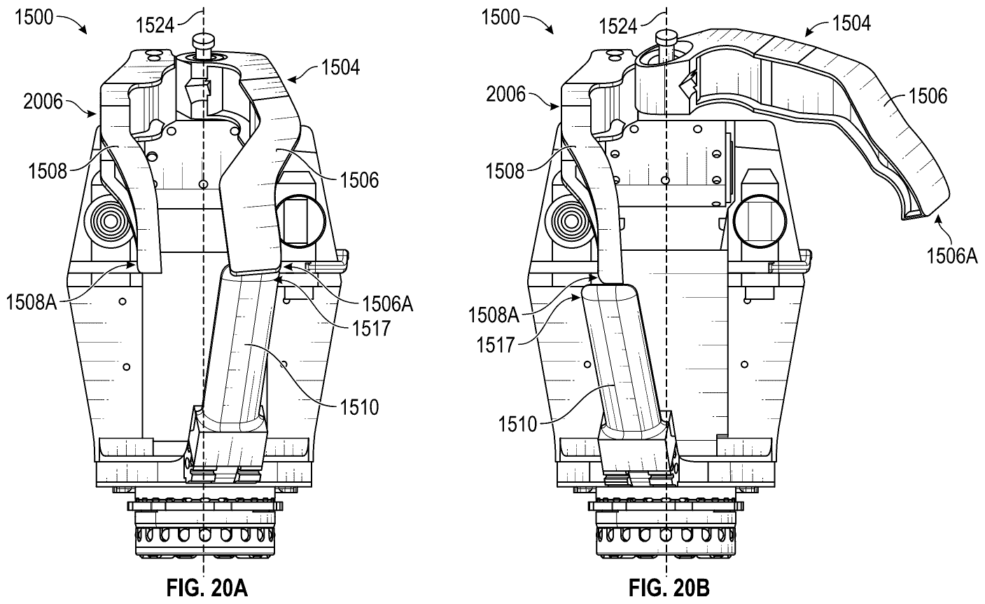

FIG. 20A illustrates the prosthetic device 1500 with the opposable thumb 1510 and a split hook 1504 of the prosthetic device 1500. The distal end 1517 of the opposable thumb 1510 is contacting a distal end 1506A of a first end effector 1506 of the split hook 1504. Accordingly, the opposable thumb 1510 and the first end effector 1506 can pinch an object between the opposable thumb 1510 and the first end effector 1506. By contacting each other, even very thin objects may be pinched, such as a piece of paper, a hair, etc.

The split hook 1504 may be positioned in an intermediate position 2006 so the distal ends 1506A, 1508A of the respective end effectors 1506, 1508 are positioned a distance from each other. The first end effector 1506 may be positioned so the distal end 1506A is off-centered from (e.g., not aligned with) the longitudinal axis 1524 or sagittal plane. The opposable thumb 1510 may be rotated about the transverse axis 1522 to any of the intermediate positions 1902, 1904, 1906 or other positions between the fully extended position 1901 (shown in FIG. 19A) and the retracted position 1804 (shown in FIGS. 18A and 18B). The opposable thumb 1510 may be positioned in the first partially abducted position 1704, the second partially abducted position 1706, the first partially adducted position 1710, and/or the second partially adducted position 1712, so the opposable thumb 1510 is off-centered from (e.g., not aligned with) the longitudinal axis 1524 and/or the sagittal axis 1701 or sagittal plane.

FIG. 20B illustrates the prosthetic device 1500 with the distal end 1517 of the opposable thumb 1510 contacting the distal end 1508A of the second end effector 1508 of the split hook 1504. Accordingly, the opposable thumb 1510 and the second end effector 1508 can pinch an object between the opposable thumb 1510 and the second end effector 1508, as described.

The split hook 1504 may be positioned in an intermediate position 2006 so the distal ends 1506A, 1508A of the end effectors 1506, 1508 are positioned a distance from each other. The second end effector 1508 may be positioned so the distal end 1508A is off-centered from (e.g., not aligned with) the longitudinal axis 1524 or sagittal plane, but opposite to the configuration shown in FIG. 20A. The opposable thumb 1510 may be positioned in any of the intermediate positions 1902, 1904, 1906 or other positions between the fully extended position 1901 (shown in FIG. 19A) and the retracted position 1804 (shown in FIGS. 18A and 18B). The opposable thumb 1510 may be positioned in the first partially abducted position 1704, the second partially abducted position 1706, the first partially adducted position 1710, and/or the second partially adducted position 1712, so the opposable thumb 1510 is off-centered from (e.g., not aligned with) the longitudinal axis 1524 or sagittal plane. For either of the configurations in FIGS. 20A and 20B, the split hook and end effectors may perform any of the movements and attain any of the positions as shown in or described with respect to FIGS. 1A-14B.

FIGS. 21A-21C illustrates the prosthetic device 1500 in a gripping position 2100. FIG. 21A illustrates a front (palm side) view of the prosthetic device 1500 in the gripping position 2100. FIG. 21B illustrates a side view of the prosthetic device 1500 in the gripping position 2100. FIG. 21C illustrates a perspective view of the prosthetic device 1500 in the gripping position 2100 and holding an elongated object embodied as a tool 2102.

As shown in FIGS. 21A and 21B, when the prosthetic device 1500 is in the gripping position 2100, the split hook 1504 may be positioned in a pinched position, such as pinched position 303, and a closed position, such as closed position 203. The opposable thumb 1510 may have been rotated about the longitudinal axis 1524 to be positioned in the first partially abducted position 1704, the second partially abducted position 1706, the first partially adducted position 1710, or the second partially adducted position 1712, or any positions therebetween. The opposable thumb 1510 may simultaneously have been rotated about the transverse axis 1522 to be positioned in any of the intermediate positions 1902, 1904, 1906 or other positions between the fully extended position 1901 (shown in FIG. 19A) and the retracted position 1804 (shown in FIGS. 18A and 18B). The opposable thumb 1510 may thus be positioned next to (e.g., adjacent to) and/or aligned with the first end effector 1506 and/or the second end effector 1508 of the split hook 1504. The opposable thumb 1510 may be on either lateral side of the split hook 1504. The configuration allows for a compact volume requiring minimal space.

As shown in FIG. 21B, the split hook 1504 and/or the opposable thumb 1510 may form a channel 2104 therebetween. The channel 2104 may extend laterally between the split hook 1504, the opposable thumb 1510 and the palm wall 1501 of the prosthetic device 1500. As shown in FIG. 21C, the tool 2102 may be positioned in the channel 2104. Since the opposable thumb 1510 is positioned next to the split hook 1504, the split hook 1504 and the opposable thumb 1510 may contact the tool 2102 at different points along the length of the tool 2102. The split hook 1504 and the opposable thumb 1510 may contact the tool 2102 at three discrete points for a stable grip. Accordingly, the split hook 1504 and the opposable thumb 1510 may secure the tool 2102 in the channel 2104 so the tool 2102 is stationary and cannot move (e.g., translate, rotate, etc.) when the tool 2102, such as a hammer, is used. The gripping position 2100 may emulate a natural gripping position of a natural hand. Therefore, the tool 2102 may be positioned similar to when the tool 2102 is gripped by a natural hand so a user can use the tool 2102 with the prosthetic device 1500 similarly to how the tool 2102 is used with a natural hand. For example, a working end (not shown) of the tool 2102 may be positioned so a user can use similar arm motions when using the tool 2102 with the prosthetic device 1500 as the user would when using the tool 2102 with a natural hand. In some embodiments, the tool 2102 may be a hammer, where the hammer is swung using the arm attached to the prosthetic device 1500. The tool 2102 may include any tool configured to be held in a natural hand, including tools not designed specifically for the prosthetic device 1500.

In some embodiments, the prosthetic device 1500 may be configured to hold the tool 2102 at a working angle. The working angle may be an angle at which the handle of the tool 2102 is positioned relative to the longitudinal axis 1524 of the prosthetic device 1500 and/or a user's forearm when the tool 2102 is used. The gripping position 2100 may allow a user to hold the tool 2102 with the prosthetic device 1500 at the working angle, for example to mimic the position the tool 2102 would naturally and functionally be held by a user in a natural human hand. In some embodiments, the working angle may be from 95 to 120 degrees, from 95 degrees to 135 degrees, from 100 to 130 degrees, from 105 to 125 degrees, or from 110 to 120 degrees. The gripping position 2100 may allow a user to hold the tool 2102 at the working angle. In some embodiments, the gripping position 2100 may allow a user to hold the tool 2102 at an angle between 100 degrees and 110 degrees. In some embodiments, the gripping position 2100 may allow a user to hold the tool 2102 at an angle from 95 to 120 degrees. In some embodiments, the gripping position 2100 may allow a user to hold the tool 2102 at an angle of 110 degrees or about 110 degrees. Accordingly, the gripping position 2100 may reduce or minimize the stress on the user's upper residual joint (e.g., elbow, shoulder) by reducing or minimizing the range of motion of the user's upper residual joint required to use the tool 2102.

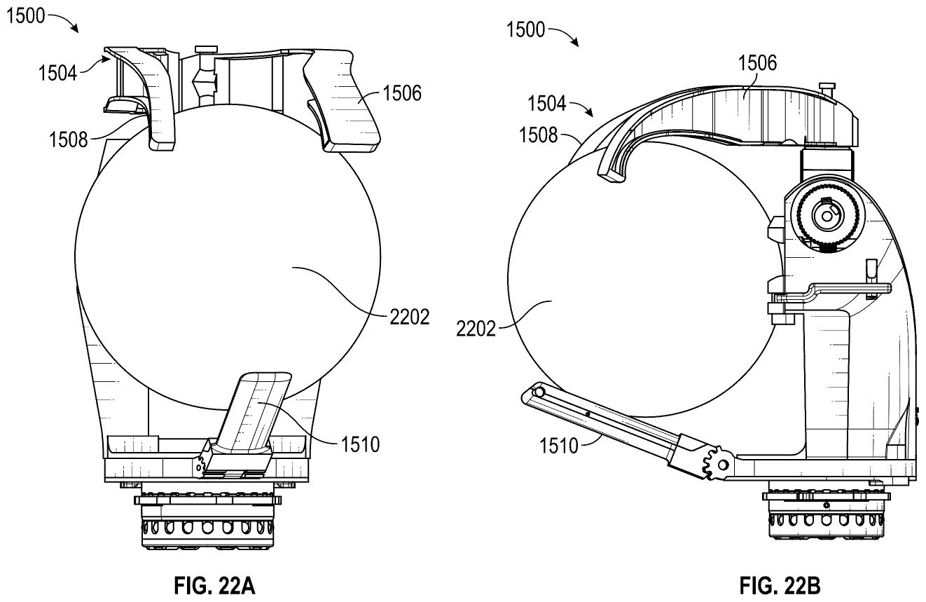

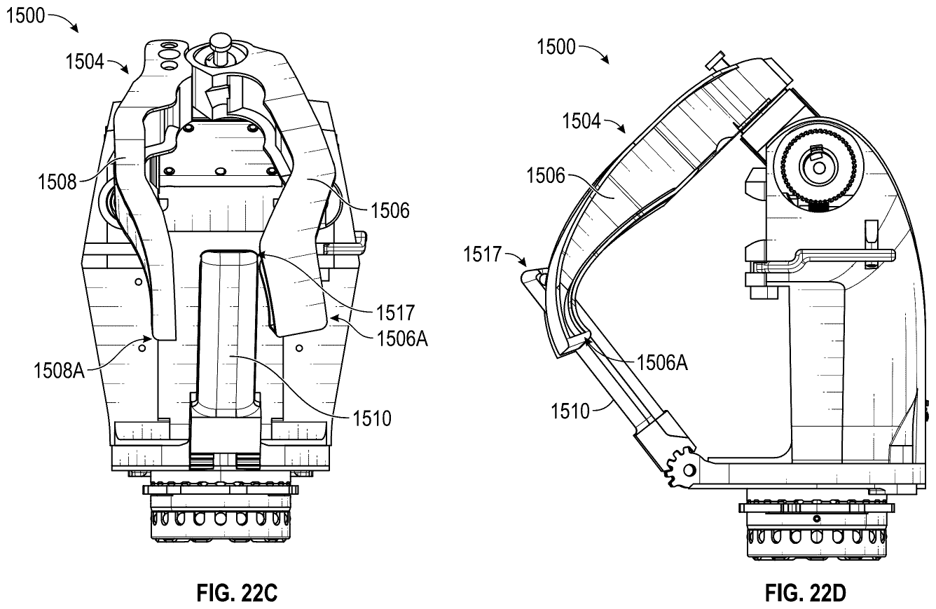

FIGS. 22A and 22B illustrate the prosthetic device 1500 with an object 2202 grasped between the split hook 1504 and the opposable thumb 1510. The split hook 1504 may be positioned in an open, unpinched position, such as the open unpinched position 702. The split hook 1504 may be positioned in an open, pinched positioned, such as the open pinched position 704. The opposable thumb 1510 may be positioned in the middle position 1708 and between the fully extended position 1901 (shown in FIG. 19A) and the retracted position 1804 (shown in FIGS. 18A and 18B). Accordingly, the opposable thumb 1510 may be centered between the first end effector 1506 and the second end effector 1508. The object 2202 may be positioned so the first end effector 1506 and second end effector 1508 of the split hook 1504 contact a top side of the object 2202, and the opposable thumb 1510 contacts a bottom side of the object 2202. The split hook 1504 and the opposable thumb 1510 may contact the tool 2102 at three discrete points for a stable grip, with the end effectors 1506, 1508 located laterally outside of the opposable thumb 1510. The object 2202 may be spherical, such as a ball or other rounded object.

As shown in FIGS. 22C and 22D, in order to hold a smaller object (not shown) than the object 2202, the opposable thumb 1510 can be positioned so the distal end 1517 of the opposable thumb 1510 is located in between the distal ends 1506A, 1508A of the end effectors 1506, 1508 of the split hook 1504. The distal end 1517 may be adjacent to the distal ends 1506A, 1508A of the end effectors 1506, 1508. The distal end 1517 and the distal ends 1506A, 1508A may be located a similar distance from the palm portion. The distal end 1517 may be located relatively closer to the palm portion than one or both of the distal ends 1506A, 1508A.

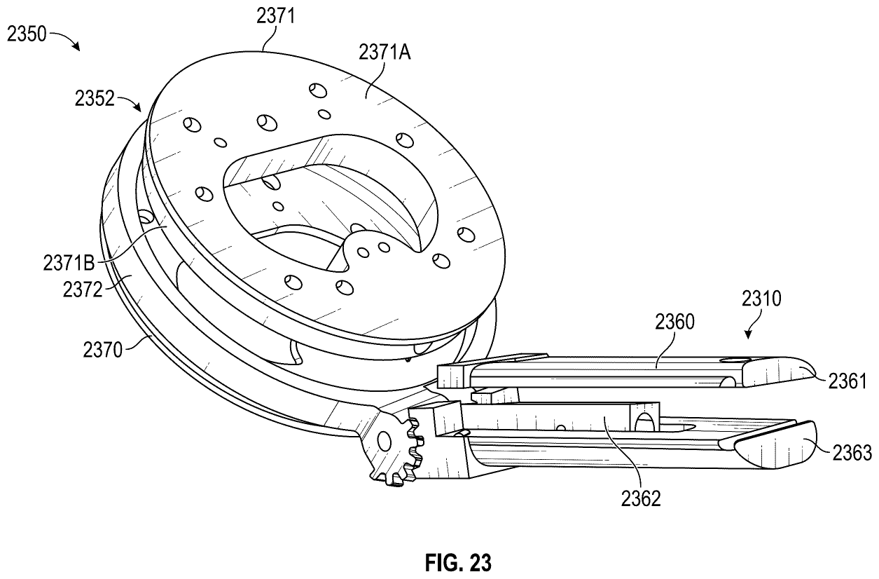

FIG. 23 illustrates a partially exploded view of another example thumb assembly 2350 for a prosthetic device, such as prosthetic device 1500. The thumb assembly 1550 may include any of the features of the thumb assembly 2350, and the thumb assembly 2350 may include any of the features of the thumb assembly 1550, except as otherwise described herein. The thumb assembly 2350 may include a rotation assembly 2352 having a bottom plate 2370 and a top plate 2371. A rotating member 2372 may be positioned between the bottom plate 2370 and the top plate 2371. The top plate 2371 is shown removed from the bottom plate 2370 and rotating member 2372 for sake of illustration. In use, the rotating member 2372 receives the top plate 2371 therein. The top plate 2371 may include a protrusion 2371B (such as a wall) extending axially from a base 2371A of the top plate 2371. The rotating member 2372 may be positioned circumferentially around the protrusion 2371B. The rotating member 2372 may be configured to rotate around the protrusion 2371B. The top plate 2371 may be coupled to a proximal end of a prosthetic device, such as the proximal end 1503 of the prosthetic device 1500, or other prosthetic devices.

The opposable thumb 2310 of the thumb assembly 2350 may include a cover 2360. The cover 2360 may be split. The cover 2360 may be longitudinally bisected into a first cover portion 2361 and a second cover portion 2363. The first cover portion 2361 and the second cover portion 2363 may be coupled together over a body 2362 of the opposable thumb 2310 in order to position the cover 2360 over the opposable thumb 2310. The second cover portion 2363 may include the rachet protrusions 2366 that interact with the rachet members 2376A of the rachet 2376 of the rotating member 2372. The rachet protrusions 2366, the rachet members 2376A, and the rachet 2376 can be the same as those parts described with reference to FIGS. 16A and 16B.

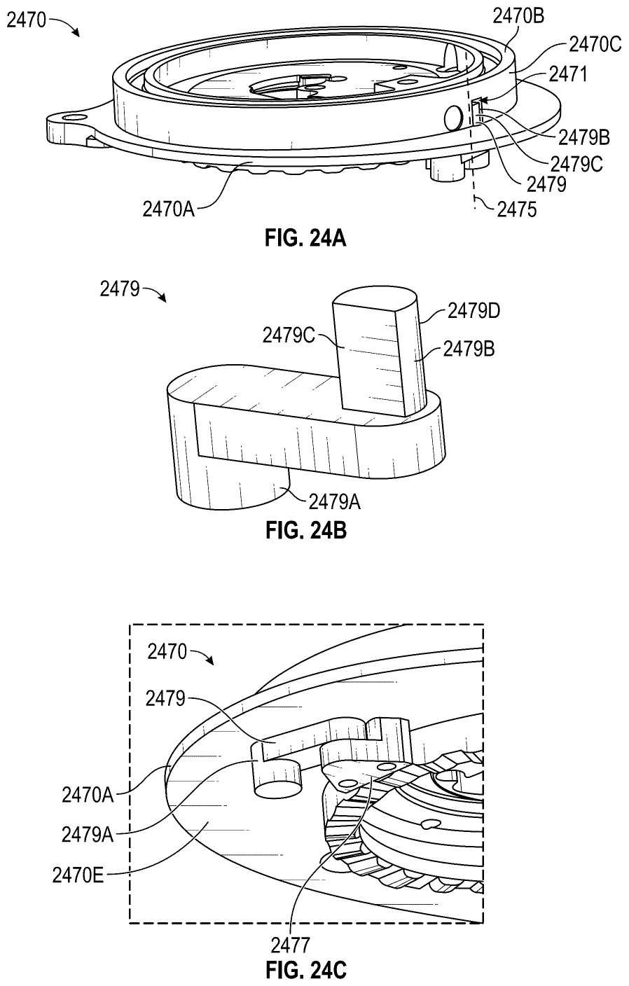

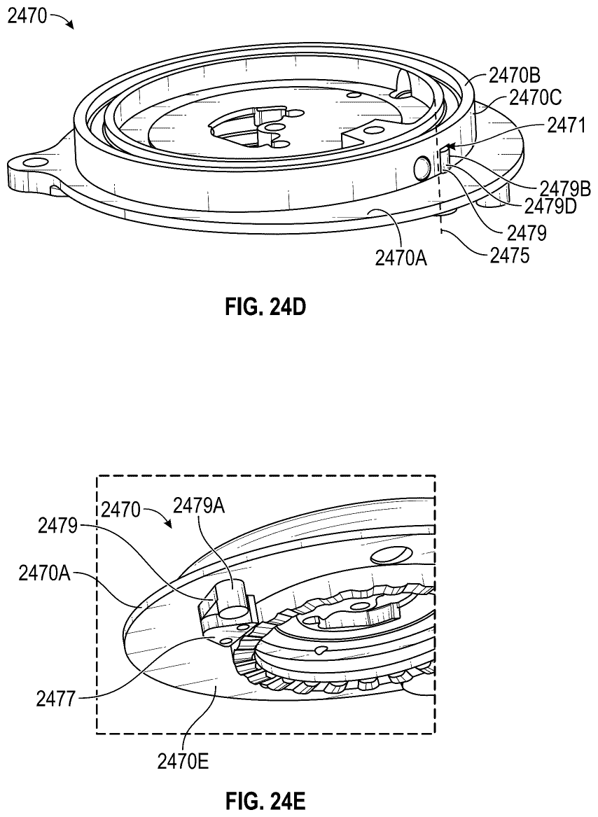

FIGS. 24A-24E illustrate features for locking and preventing rotation of the rotation assembly (as opposed to just providing a securement force which may be overcome with sufficient rotational force, as in the ball and detent mechanism). Another example plate 2470 is shown for a rotation assembly of a prosthetic device, such as the rotation assembly 1552 of the prosthetic device 1500. The plates 1570, 2370, 2371 may include any of the features of the plate 2470, and vice versa, except as otherwise described herein. FIG. 24A illustrates a top perspective view of the plate 2470 with a locking cam 2479 in an unlocked position. FIG. 24B illustrates the locking cam 2479 in isolation. FIG. 24C illustrates a partial bottom perspective view of the plate 2470 with the locking cam 2479 in the unlocked position. FIG. 24D illustrates a top perspective view of the plate 2470 with the locking cam 2479 in a locked position. FIG. 24E illustrates a partial bottom perspective view of the plate 2470 with the locking cam 2479 in the locked position.

As shown in FIG. 24A, the plate 2470 can include an opening 2471 in the protrusion 2470B of the plate 2470. The plate 2470 can include a locking cam (e.g., locking mechanism) 2479 positioned in the opening 2471. The locking cam 2479 can be configured to rotate about an axis 2475 from an unlocked position, shown in FIGS. 24A and 24C, to a locked position, shown in FIGS. 24D and 24E. The axis 2475 may be an axis extending through a cam portion 2479B of the locking cam 2479 perpendicular to the base 2470A of the plate 2470.

As shown in FIG. 24B, the locking cam 2479 may include a lever portion 2479A and a cam portion 2479B extending from the lever portion 2479A. The cam portion 2479B may include a first side 2479C and a second side 2479D. The first side 2479C of the cam portion 2479B may be flat and the second side 2479D of the cam portion 2479B may be rounded.

As shown in FIGS. 24A and 24D, the cam portion 2479B may be positioned in the opening 2471 of the plate 2470. When the locking cam 2479 is in the unlocked position, shown in FIG. 24A, the cam portion 2479B may be positioned so the first side 2479C of the cam portion 2479B faces outward from the opening 2471. Since the first side 2479C is flat, the cam portion 2479B may not extend outward beyond the outer surface 2470C of the protrusion 2470B of the plate 2470.

As shown in FIGS. 24C and 24D, the lever portion 2479A may be positioned on a bottom side 2470E of the base 2470A of the plate 2470. A user may rotate the lever portion 2479A in order to rotate the locking cam 2479 between the unlocked position and the locked position. The plate 2470 may include a bracket 2477. The bracket 2477 may be coupled to the bottom side 2470E of the base 2470A of the plate 2470. The bracket 2477 may be configured to secure the locking cam 2479 in place so the cam portion 2479B is positioned in the opening 2471. The bracket 2477 may allow the locking cam 2479 to rotate between the unlocked position and the locked position. As shown in FIG. 24C, when the locking cam 2479 is in the unlocked position, the lever portion 2479A may extend in a lateral direction from the bracket 2477

As shown in FIG. 24D, when the locking cam 2479 is in the locked position, the second side 2479D of the cam portion 2479B faces outward from the opening 2471. Since the second side 2479D is rounded, the cam portion 2479B may extend outward beyond the outer surface 2470C of the protrusion 2470B of the plate 2470. Accordingly, the cam portion 2479B may extend into a recess in a rotating member, such as rotating member 1572, in order to prevent or inhibit rotation of the rotating member. As shown in FIG. 24E, when the locking cam 2479 is in the locked position, the lever portion 2479A may extend in a medial direction from the bracket 2477.

FIG. 25A-25H illustrate certain details of the release mechanism 1590 of the prosthetic device 1500. FIG. 25A illustrates a cross-section of the prosthetic device 1500 with the release mechanism 1590 in an engaged position. FIG. 25B illustrates a cross-section of the prosthetic device 1500 with the release mechanism 1590 in a disengaged position. FIGS. 25C-25E illustrate partial cross-sectional views of the prosthetic device 1500 with the release mechanism 1590 in the engaged position. FIG. 25F illustrates a partial cross-sectional view of the prosthetic device 1500 with the release mechanism 1590 in the disengaged position. FIGS. 25G and 25H illustrate perspective views of a cam 1597 of the release mechanism 1590 in isolation.

As shown in FIG. 25A, when the release mechanism 1590 is in the engaged position, the driven gears 1596 and the worm gears 1594 of the drive assembly 1592 may be engaged, and the lever arm 1591 of the release mechanism 1590 may extend toward a dorsal side 1507 of the prosthetic device 1500. As shown in FIG. 25B, when the release mechanism 1590 is in the disengaged position, the driven gears 1596 and the worm gears 1594 of the drive assembly 1592 may be disengaged, and the lever arm 1591 of the release mechanism 1590 may extend toward the palm side 1509 of the prosthetic device 1500.

As shown in FIGS. 25C-25F, the worm gear 1594 may be rotatably supported by a first ball bearing assembly 1595A and a second ball bearing assembly 1595B. The first ball bearing assembly 1595A may be coupled to a first (e.g., dorsal) end 1594A of the worm gear 1594 and the second ball bearing assembly 1595B may be coupled to a second (e.g., palm) end 1594B of the worm gear 1594. The second ball bearing assembly 1595B and the second end 1594B of the worm gear 1594 may be positioned in bearing cup 1595C. The bearing cup 1595C may be configured to support the second ball bearing assembly 1595B and the second end 1594B of the worm gear 1594.

The release mechanism 1590 may include a cam 1597. The cam 1597 may be coupled to the housing 1511 of the prosthetic device 1500 so the cam 1597 extends along the axis 1593 and perpendicular to a longitudinal axis 1598 of the worm gear 1594. The longitudinal axis 1598 may be an axis extending between the first end 1594A of the worm gear 1594 and the second end 1594B of the worm gear 1594.

As shown in FIGS. 25G and 25H, the cam 1597 may include a body 1597A. The body 1597A may include an elongated shape. The body 1597A may be cylindrical shaped. The body 1597A may include a recess 1597B extending transversely into the body 1597A. The recess 1597B may be sized and/or shaped so the bearing cup 1595C fits into the recess 1597B.

As shown in FIGS. 25C-25E, when the release mechanism 1590 and/or the cam 1597 is in the engaged position, the cam 1597 may be positioned so the recess 1597B faces the palm side 1509 of the prosthetic device 1500 (e.g., away from the bearing cup 1595C) and the body 1597A contacts the bearing cup 1595C. The cam 1597 may be configured to prevent or inhibit the second ball bearing assembly 1595B, the bearing cup 1595C, and/or the worm gear 1594 from translating along the longitudinal axis 1598 of the worm gear 1594 towards the palm side 1509 of the prosthetic device 1500. For example, the cam 1597 may prevent or inhibit the worm gear 1594 from translating along the longitudinal axis 1598 when an external force in applied to the split hook 1504 of the prosthetic device 1500.

As shown in FIG. 25F, when the release mechanism 1590 and/or the cam 1597 is in the disengaged position, the cam 1597 may be positioned so the recess 1597B faces the dorsal side 1507 of the prosthetic device 1500 (e.g., towards the bearing cup 1595C). Accordingly, bearing cup 1595C may translate into the recess 1597B, and, the second ball bearing assembly 1595B, the bearing cup 1595C, and/or the worm gear 1594 may translate along the longitudinal axis 1598 towards the palm side 1509 of the prosthetic device 1500. When the worm gear 1594 translates toward the palm side 1509 of the prosthetic device 1500, teeth of the driven gear 1596 may not contact the teeth of the worm gear 1594. The worm gear 1594 may be translated toward the palm side 1509 of the prosthetic device 1500 by applying a force (e.g., rotating) the split hook 1504. When the split hook 1504 is rotated, the driven gear 1596 may rotate and the rotation of the driven gear 1596 may cause the worm gear 1594 to translate towards the palm side 1509 of the prosthetic device 1500 until the teeth of the worm gear 1594 disengage from the teeth of the driven gear 1596.

As shown in FIG. 25C, the lever arm 1591 may be coupled to the cam 1597. The lever arm 1591 may be configured to rotate the cam 1597 about the axis 1593. As described above, when the release mechanism 1590 and/or the cam 1597 is in the engaged position, the lever arm 1591 may extend towards the dorsal side 1507 of the prosthetic device 1500, and the recess 1597B of the cam 1597 may face towards the palm side 1509 of the prosthetic device 1500 (e.g., away from the bearing cup 1595C).

When the lever arm 1591 is rotated about the axis 1593, the lever arm 1591 may rotate the cam 1597 about the axis 1593 to the disengaged position. As described above, and shown in FIG. 25F, when the release mechanism 1590 and/or the cam 1597 is in the disengaged position, the lever arm 1591 may extend towards the palm side 1509 of the prosthetic device 1500, and the recess 1597B of the cam 1597 may face towards the dorsal side 1507 of the prosthetic device 1500 (e.g., towards the bearing cup 1595C).

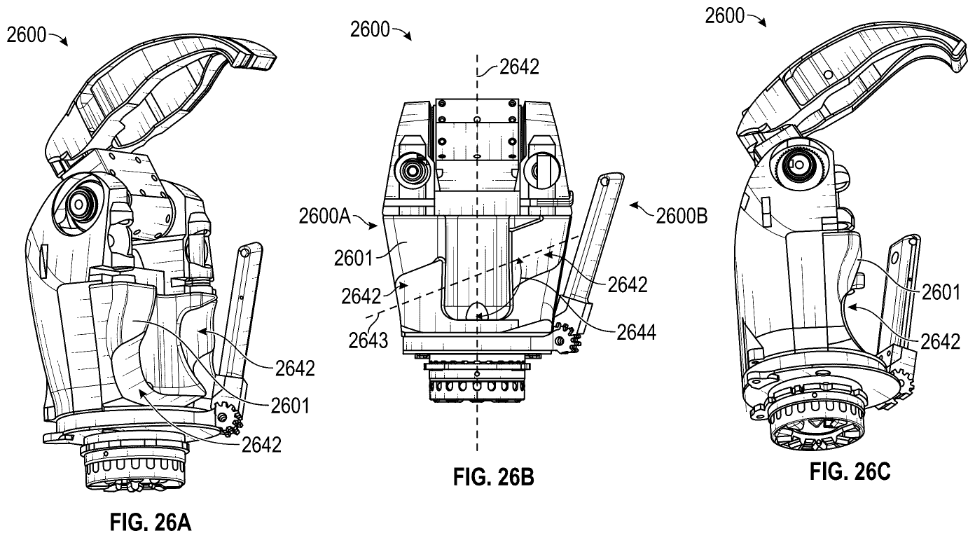

FIGS. 26A-26C illustrate another example prosthetic assistive hand device or “prosthetic device” 2600 and features thereof. The prosthetic device 100, 1500 may include any of the features of the prosthetic device 2600, and vice versa, except as otherwise described herein. FIG. 26A illustrates a bottom-front perspective view of the prosthetic device 2600. FIG. 26B illustrates a front (e.g., palm side) view of the prosthetic device 2600. FIG. 26B illustrates a bottom-side perspective view of the prosthetic device 2600.

The palm wall 2601 of the prosthetic device 2600 may include a channel 2642. As shown in FIG. 26B, the channel 2642 may extend across the palm wall 2601 at an angle with the longitudinal axis 2624 of the prosthetic device 2600. An axis 2643 extending through the channel 2642 from a first (lateral) side 2600A of the prosthetic device 2600 to a second (medial) side 2600B of the prosthetic device 2600 may extend at an angle 2644 with the longitudinal axis 2624. The angle 2644 may be 80 degrees, 85 degrees, 90 degrees, 95 degrees, 100 degrees, 105 degrees, 110 degrees, 115 degrees, 120 degrees, 125 degrees, 130 degrees, 135 degrees, 140 degrees, and/or any value between the aforementioned values. In some embodiments, the angle 2644 may be between 80 degrees and 140 degrees. In some embodiments, the angle 2644 may be between 95 degrees and 120 degrees. In some embodiments, the angle 2644 may be between 100 degrees and 110 degrees. In some embodiments, the angle 2644 may be between 110 degrees and 120 degrees.

The channel 2642 may be sized and/or shaped to receive an object, such as a tool. Accordingly, when the prosthetic device 2600 is in a gripping position, such as gripping position 2100 described with reference to FIGS. 21A-21C, the channel 2642 may prevent or inhibit movement of the object in order to secure the object at the angle 2644.

The prosthetic device 1500 therefore provides for numerous advantages. For example, seamless transitions between configurations using a spring-return mechanism are possible, making it easier for users, including those with bilateral devices, to deploy and engage the thumb. The thumb can be deployed by rotating the thumb from an “in-between parked position” (with the thumb perpendicular to the sagittal axis and fully flexed) or flexion and extension can deploy the thumb by pulling the phalange of the thumb (no button required, requires only grasping the thumb). The in-between parked position provides a concession between the aesthetic option of fully parked thumb in the sagittal plan, and fully deployed thumb that is functional only in a 3 jaw chuck option—this in between solution offers a fast re-engagement of the thumb for more versatility when transitioning often between split hook option and 3-jaw chuck option. The prosthetic device 1500 may provide an optimized palm surface utilization. Repositioning the thumb may maximize palm usability, enhancing functional interactions with objects (such as cylindrical grasp, spherical grasp, and handle of tools). The prosthetic device 1500 may provide an improved line of sight. The thumb's position minimizes obstruction, providing a clearer view for precise control. The prosthetic device 1500 may provide a more streamlined design. A more compact thumb may offer the cylindrical span of 100 mm but at a reduced length compared to the prior art, reducing bulk and enhancing the ability to reach in-between, confined spaces. The prosthetic device 1500 may provide for enhanced lateral positioning. The device allows effective power grips for tool handling by facilitating proper tool alignment with the forearm's axis, improving overall functionality. The rotation of the thumb may use a more refined activation mechanism that requires less effort, ensuring ease of use and quick transitions for thumb deployment and reployment. The prosthetic device 1500 may provide for better safety and cutlery stability improvements. The thumb need not create a sharp angle with the palm thus avoiding any possibility to trap an object unintentionally between the palm and the thumb. Additionally, the disc creates a support for cutlery stabilization, with reinforced grip stability between split hooks.

Various modifications to the implementations described in this disclosure can be readily apparent to those skilled in the art, and the generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “example” is used exclusively herein to mean “serving as an example, instance, or illustration.” Any implementation described herein as “example” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results, except as otherwise described. In some cases, the actions recited in the claims can be performed in a different order and still achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

What is claimed is:

1. A prosthetic assistive device comprising:
a palm portion extending along a longitudinal axis and configured to attach to a distal end of a natural or prosthetic arm or wrist;
a split hook comprising a first end effector and a second end effector, wherein the first end effector and the second end effector are configured to rotate about a first axis to cause a pinching or unpinching rotation, and wherein the first and/or second end effectors are configured to rotate about a second axis perpendicular to the first axis to open or close relative to each other;
a drive assembly configured to cause the split hook to rotate about the first and second axes; and
a thumb assembly coupled to a proximal end of the palm portion, the thumb assembly comprising a third end effector, wherein the third end effector is configured to rotate about a transverse axis to move between a retracted position and an extended position, and wherein the third end effector is configured to rotate about the longitudinal axis to adduct and abduct.

2. The prosthetic assistive device of claim 1, wherein the third end effector is configured to rotate to a plurality of discrete positions between the retracted position and the extended position.

3. The prosthetic assistive device of claim 1, wherein the third end effector is configured to rotate about the longitudinal axis to a plurality of discrete positions between an abducted position and an adducted position.

4. The prosthetic assistive device of claim 1, wherein the thumb assembly further comprises a rotation assembly coupled to the proximal end of the palm portion, wherein the third end effector is coupled to the rotation assembly, and wherein the rotation assembly is configured to rotate about the longitudinal axis to rotate the third end effector about the longitudinal axis.

5. The prosthetic assistive device of claim 4, wherein the rotation assembly comprises a locking mechanism configured to prevent or inhibit rotation of the rotation assembly when the locking mechanism is in a locked position.

6. The prosthetic assistive device of claim 4, wherein the rotation assembly further comprises:
a plate coupled with the proximal end of the palm portion; and
an annular rotating member rotatably coupled with the plate and the palm portion.

7. The prosthetic assistive device of claim 6, wherein the third end effector is rotatably coupled to the annular rotating member.

8. The prosthetic assistive device of claim 6, wherein the rotating member comprises a ratchet member, and the third end effector is rotatably coupled to the ratchet member.

9. The prosthetic assistive device of claim 8, wherein the ratchet member is configured to maintain the end effector in a plurality of discrete positions between the retracted position and the extended position.

10. The prosthetic assistive device of claim 8, wherein the third end effector comprises a cover slidably positioned over a body.

11. The prosthetic assistive device of claim 10, wherein the third end effector comprises a biasing member configured to bias the cover of the third end effector towards the ratchet member.

12. The prosthetic assistive device of claim 1, further comprising a release mechanism configured to disengage the drive assembly.

13. The prosthetic assistive device of claim 12, wherein the release mechanism comprises a lever and a cam, wherein the lever is configured to rotate the cam between an engaged position and a disengaged position.

14. The prosthetic assistive device of claim 13, wherein in the engaged position a recess in the cam faces away from a worm gear of the drive assembly and the cam prevents or inhibits translation of a worm gear of the drive assembly, and wherein in the disengaged position, the recess in the cam faces the worm gear of the drive assembly so the worm gear translates into the recess of the cam.

15. The prosthetic assistive device of claim 5, wherein the locking mechanism comprises a cam configured to rotate between the locked position and an unlocked position.

16. The prosthetic assistive device of claim 6, wherein the rotation assembly comprises a detent mechanism positioned in the plate, wherein the detent mechanism is configured to extend into a recess of a plurality of recesses in the annular rotating member in order to maintain the third end effector in one of a plurality of discrete positions between an abducted position and an adducted position.

17. The prosthetic assistive device of claim 16, wherein the detent mechanism comprises a ball and spring.

18. A prosthetic assistive device configured to attach to a distal end of a natural or prosthetic arm or wrist, the prosthetic assistive device comprising:
a split hook comprising a first end effector and a second end effector; and
a drive assembly comprising a pair of motors, a pair of motor gears coupled to the motors, a pair of worm gear assemblies, a pair of driven gear assemblies, and a bevel gear, wherein the pair of worm gear assemblies are positioned so an axis of rotation of the pair of worm gear assemblies extends from a dorsal side of the prosthetic device to a palm side of the prosthetic device, and
wherein the drive assembly is configured to rotate the split hook about two axes.

19. The prosthetic assistive device of claim 18, wherein the motors are configured to rotate the pair of driven gear assemblies in a same first direction to cause a pinch rotation of the split hook, and to rotate the pair of driven gear assemblies in a same second direction opposite the first direction to cause an unpinch rotation of the split hook.

20. The prosthetic assistive device of claim 18, further comprising a release mechanism configured to disengaged the drive assembly.
